(19) United States
(12) Patent Application Publication
(10) Pub. No.: US 2024/0374542 A1
(43) Pub. Date: Nov. 14, 2024

(54) ORAL DOSAGE FORMS OF METFORMIN AND PREPARATION METHOD THEREOF

(71) Applicant: ELITE PHARMACEUTICAL SOLUTION INC., Princeton, NJ (US)

(72) Inventors: Wu TIAN, Princeton, NJ (US); Yan WANG, Princeton, NJ (US); Henry TIAN, Princeton, NJ (US)

(73) Assignee: ELITE PHARMACEUTICAL SOLUTION INC., Princeton, NJ (US)

(21) Appl. No.: 18/195,890

(22) Filed: May 10, 2023

(51) Int. Cl. A61K 31/155; A61K 9/00; A61K 9/20; A61K 9/28

(52) U.S. Cl. CPC A61K 31/155; A61K 9/0053; A61K 9/2013; A61K 9/2027; A61K 9/2054; A61K 9/2853; A61K 9/2866

(57) ABSTRACT

An oral dosage form of metformin and preparation method is provided. The oral dosage form comprises a metformin-containing core and an encapsulating controlled membrane film, and the controlled membrane film is provided with at least one passageway allowing metformin to release out of the core therethrough in an aqueous environment. The oral dosage form is designed such that upon dissolving in a medium with a pH 6.8 at 37° C., less than 30% of metformin is released at 4 hours, and less than 92% of the metformin is released at 24 hours. The oral dosage form is further designed to provide a maximum plasma concentration of the metformin or the pharmaceutically acceptable salt thereof in the subject from approximately 8-24 hours after a single-dose oral administration.

### TECHNICAL FIELD

The present disclosure is directed generally to pharmaceutical compositions for treating Type 2 diabetes, and more specifically to oral dosage forms of an antidiabetic agent, such as metformin. The oral dosage forms substantially comprise a controlled release form of metformin or a pharmaceutically acceptable salt thereof, designed such that metformin is released in a relatively less amount in the upper gastrointestinal (GI) tract and is released in a relatively more amount in the down gastrointestinal (GI) tract, thereby realizing a more consistant dosage release at different release time points. This present disclosure is further directed to processes for preparing such oral dosage forms, and to methods for treating Type 2 diabetes with such oral dosage forms.

### BACKGROUND

Type 2 diabetes, also known as hyperglycemia, is a chronic and progressive disease that has been identified as a world epidemic affecting approximately 9% of the world population. Type 2 diabetes is primarily caused by endocrine defects or deficiencies such as insulin resistance and impaired insulin secretion.

Treatment of Type 2 diabetes typically begins with diet and exercise, followed by oral antidiabetic monotherapy. Currently there are a variety of types/classes of antidiabetic medications that can be administered for oral antidiabetic monotherapy, including biguanides, dipeptidyl peptidase 4 (DPP-4) inhibitors, sulfonylureas, meglitinides, thiazolidinediones, sodium-glucose transporter 2 (SGLT2) inhibitors, and alpha-glucosidase inhibitors, etc.

As an approved first line of antidiabetic agent, metformin is a biguanide that improves glucose tolerance in patients with Type 2 diabetes, lowering both basal and postprandial plasma glucose. Metformin decreases hepatic glucose production, decreases intestinal absorption of glucose, and improves insulin sensitivity by increasing peripheral glucose uptake and utilization. Metformin does not produce hypoglycemia in patients with type 2 diabetes or in healthy subjects except in special circumstances and does not cause hyperinsulinemia. With metformin therapy, insulin secretion remains unchanged while fasting insulin levels and daylong plasma insulin response may decrease.

In one notable example, metformin salts, and notably metformin hydrochloride, are typically highly water-soluble which can, if administered in an uncontrolled manner, frequently cause gastrointestinal (GI) side effects, such as diarrhea, nausea, and vomiting, occurring more than all other oral antidiabetic agents, yet also has limited duration of pharmaceutical effects, given its PK and PD profiles. Although these GI side effects can diminish over time and can be minimized by taking metformin at mealtimes or by careful dose adjustment, they may impair compliance and even lead to discontinued therapy for certain patients. Similar gastrointestinal side effects were observed with some other antidiabetic agents as well.

Although metformin has become a drug of choice for the treatment of type 2 diabetes mellitus, some patients may not receive it owing to the risk of lactic acidosis. Metformin, along with other drugs in the biguanide class, increases plasma lactate levels in a plasma concentration-dependent manner by inhibiting mitochondrial respiration predominantly in the liver. Elevated plasma metformin concentrations (as occur in individuals with renal impairment) and a secondary event or condition that further disrupts lactate production or clearance (e.g., cirrhosis, sepsis, or hypoperfusion), are typically necessary to cause metformin-associated lactic acidosis (MALA). As these secondary events may be unpredictable and the mortality rate for MALA approaches 50%, metformin has been contraindicated in moderate and severe renal impairment since its FDA approval in patients with normal renal function or mild renal insufficiency to minimize the potential for toxic metformin levels and MALA. An investigational delayed-release metformin currently under development could potentially provide a treatment option for patients with renal impairment pending the results of future studies.

In view of these issues, several patent documents, including U.S. Pat. Nos. 4,915,952, 5,328,942, 5,451,409, 5,945,125, 6,090,411, 6,210,710, 6,217,903, 6,488,962, and 6,723,340, 6,866,866 and 8,323,692, and International Patent Application Nos.: WO1996026718A2 and WO1997018814A1, have disclosed controlled-release, extended-release, or prolonged-release drug dosage forms of metformin hydrochloride. Such prolonged or controlled release is realized either through limiting the rate by which the surrounding gastric fluid can diffuse through the matrix and reach the drug, dissolve the drug, and diffuse out again with the dissolved drug, or through using a matrix that slowly erodes, thereby continuously exposing fresh drug to the surrounding fluid. As such, the medicine realizes a controlled release into at least a portion within the body defined by the stomach and the upper gastrointestinal (GI) tract, thereby providing continuous and non-pulsating therapeutic levels of metformin hydrochloride to human subjects in need of such treatment over a twelve-hour to twenty-four-hour period.

In the prior art, many techniques have been used to provide controlled and extended-release pharmaceutical dosage forms in order to maintain therapeutic serum levels of medicaments and to minimize the effects of missed doses of drugs caused by a lack of patient compliance and reduce side effect by reducing the possibility of dumping of drug in gastrointestinal GI system or reducing the over exposing the drug on the surface of the gastrointestinal GI system.

Although vast amounts of research has been performed on controlled or sustained release compositions and in particular on osmotic dosage forms, the side effects are still observed from the patient with the current markeked products, and difference of the release rate at variable time point among the tablets from the current marketd products is remarkable high, which could cause the dosage dumping, and futher cause the servious side effect. So further reducing side effect and proving a metformin dosage form for the patients with moderate and severe renal impairment is still needed.

### SUMMARY OF THE INVENTION

In a first aspect, the present disclosure provides an oral dosage form of a pharmaceutical composition for managing diabetes or prediabetes in a subject.

The oral dosage form substantially comprises a core porton (i.e. core tablet or core) and a controlled membrane film encapsulating the core portion. The core portion comprises metformin or a pharmaceutically acceptable salt thereof, which can optionally be designed at a therapeutically effective amount. The controlled membrane film is provided with at least one passageway configured to allow metformin or its pharmaceutically acceptable salt to release out of the core therethrough when the oral dosage form is in an aqueous environment, such as in water or in the gastrointestinal (GI) tract of the subject.

The oral dosage form is configured to realize a controlled release of metformin. Specifically, the oral dosage form can be designed to have a dissolution profile such that upon dissolving in a medium with a pH of approximately 6.8 at approximately 37° C., less than 30% of the metformin or the pharmaceutically acceptable salt thereof is released from the oral dosage form at 4 hours, and less than 92% of the metformin or the pharmaceutically acceptable salt thereof is released from the oral dosage form at 24 hours.

The oral dosage form can be further optionally designed to have a dissolution profile such that upon dissolving in a medium with a pH of approximately 6.8 at approximately 37° C., approximately 30%-50% of the metformin or the pharmaceutically acceptable salt thereof is released from the oral dosage form at 8 hours.

Further optionally, the oral dosage form can be designed to have dissolution profile such that upon dissolving in a medium with a pH of approximately 6.8 at approximately 37° C., approximately 45%-70% of the metformin or the pharmaceutically acceptable salt thereof is released from the oral dosage form at 12 hours.

The oral dosage form can be further configured to optionally have a dissolution profile such that upon dissolving in a medium with a pH of approximately 6.8 at approximately 37° C., approximately 18-21% of the metformin or the pharmaceutically acceptable salt thereof is released from the oral dosage form at 4 hours, and approximately 82-90% of the metformin or the pharmaceutically acceptable salt thereof is released from the oral dosage form at 24 hours. Further optionally, the oral dosage form can be configured to have a dissolution profile such that upon dissolving in a medium with a pH of approximately 6.8 at approximately 37° C., approximately 35%-45% of the metformin or the pharmaceutically acceptable salt thereof is released from the oral dosage form at 8 hours. Further optionally, the oral dosage form can be configured to have a dissolution profile such that upon dissolving in a medium with a pH of approximately 6.8 at approximately 37° C., approximately 50%-65% of the metformin or the pharmaceutically acceptable salt thereof is released from the oral dosage form at 12 hours.

In any of the oral dosage forms as disclosed above, at a time point between 4 hours and 24 hours after dissolving in a medium with a pH of approximately 6.8 at approximately 37° C., release of the metformin or the pharmaceutically acceptable salt thereof from the oral dosage form can have a relative standard deviation (RSD) of no more than 11%, and preferably of no more than 6%.

In any of the oral dosage forms as disclosed above, at 8 hours after dissolving in a medium with a pH of approximately 6.8 at approximately 37° C., release of the metformin or the pharmaceutically acceptable salt thereof from the oral dosage form can have a relative standard deviation (RSD) of no more than 7.5%.

In any of the oral dosage forms as disclosed above, at 12 hours after dissolving in a medium with a pH of approximately 6.8 at approximately 37° C., release of the metformin or the pharmaceutically acceptable salt thereof from the oral dosage form has a relative standard deviation (RSD) of no more than 6.0%.

In any of the oral dosage forms as disclosed above, the oral dosage form can be configured to further realize a controlled release of the metformin or the pharmaceutically acceptable salt thereof such that upon a single-dose oral administration, the oral dosage form provides a maximum plasma concentration of the metformin or the pharmaceutically acceptable salt thereof in the subject from approximately 8 to 24 hours after administration.

Herein optionally, upon a single-dose oral administration, the oral dosage form may provide a maximum plasma concentration of the metformin or the pharmaceutically acceptable salt thereof in the subject at a mean of approximately 13.5 hours and standard deviation of approximately 4.4 hours after administration.

In any of the oral dosage forms as disclosed above, the oral dosage form can be configured to realize a controlled release of the metformin or the pharmaceutically acceptable salt thereof such that upon a single-dose oral administration, the oral dosage form provides a mean maximum plasma concentration (Cₘₐₓ) of metformin from approximately 0.5*X ng/ml to approximately 0.9*X ng/ml, based on administration of the oral dosage form comprising X mg of metformin HCl, wherein X is in a range of approximately 100-1000.

In any of the oral dosage forms as disclosed above, the oral dosage form can be configured to provide a mean maximum AUC₀₋ₜ from approximately 7*Y hr*ng/ml to approximately 16*Y hr*ng/mL, based on administration of the oral dosage form comprising Y mg of metformin HCl, wherein Y is in a range of approximately 100-1000.

In any of the oral dosage forms as disclosed above, the controlled membrane film comprises at least one water insoluble polymer, each selected from a group consisting of a cellulose ester, a cellulose diester, a cellulose triester, a cellulose ether, a cellulose ester-ether, cellulose acylate, cellulose diacylate, cellulose triacylate, cellulose acetate, cellulose diacetate, cellulose triacetate, cellulose acetate propionate, and cellulose acetate butyrate. According to some embodiments of the oral dosage form, the at least one water insoluble polymer in the controlled membrane film comprises cellulose acetate, which can optionally have an acetyl content of approximately 39.3%-40.3%.

In the oral dosage form, the controlled membrane film can further comprise at least one pore-forming agent, each selected from a group consisting of sodium chloride, potassium chloride, sucrose, sorbitol, mannitol, polyethylene glycol (PEG), propylene glycol, hydroxypropyl cellulose, hydroxypropyl methylcellulose, hydroxypropyl methylcellulose phthalate, cellulose acetate phthalate, polyvinyl alcohol, and a methacrylic acid copolymer. According to some embodiments of the oral dosage form, the at least one pore-forming agent in the controlled membrane film comprises polyethylene glycol (PEG).

In any of the oral dosage forms as disclosed above, the controlled membrane film can comprise a weight ratio of 1.8-6.0%.

In any of the oral dosage forms as disclosed above, the number of the at least one passageway in the controlled membrane film is two.

Herein optionally, the two passageways can be arranged on opposing sides of oral dosage form.

Herein optionally, each of the at least one passageway can have a diameter of approximately 0.30-2.00 mm.

Herein optionally, each of the at least one passageway can have a depth of approximately 0.10-2.00 mm.

In any of the oral dosage forms as disclosed above, the metformin or a pharmaceutically acceptable salt thereof comprises at least one of metformin hydrochloride, metformin sulfate, metformin phosphate, metformin hydrobromide, metformin salicylate, metformin maleate, metformin benzoate, metformin succinate, metformin ethanesulfonate, metformin fumarate, or metformin glycolate.

According to some embodiments, the metformin or a pharmaceutically acceptable salt thereof comprises metformin hydrochloride (metformin HCl), which can have a dosage of approximately 100-1200 mg.

In any of the oral dosage forms as disclosed above, the core portion can further comprise at least one binder, each selected from a group consisting of polyvinyl pyrrolidone, hydroxypropyl cellulose, hydroxyethyl cellulose, ethylcellulose, polymethacrylate and wax.

According to some embodiments, the at least one binder in the core portion comprises polyvinyl pyrrolidone, hydroxypropyl cellulose, or a combination thereof.

According to some embodiments, the at least one binder in the core portion comprises hydroxypropyl cellulose.

According to some embodiments, the at least one binder in the core portion comprises polyvinyl pyrrolidone having an average molecular weight of 25,000 to 3,000,000 g/mol.

In any of the oral dosage forms as disclosed above, the core portion further comprises at least one absorption enhancer, each selected from a group consisting of a fatty acid, a surfactant, a chelating agent and a bile salt.

According to some embodiments, the at least one absorption enhancer in the core portion comprises a surfactant, selected from a group consisting of sodium lauryl sulfate, sodium taurocholate, and polyoxyethylene 20 sorbitan monooleate (i.e. TWEEN 80). Herein, according to some embodiments, the at least one absorption enhancer in the core portion comprises sodium lauryl sulfate.

In any of the oral dosage forms as disclosed above, the core portion further comprises at least one lubricant, each selected from a group consisting of magnesium stearate, stearic acid, sodium fumarate, glyceryl behenate, and glyceryl dibehenate.

Herein, according to some embodiments, the at least one lubricant in the core portion consists of magnesium stearate, glyceryl dibehenate, or a combination thereof. Herein optionally, the at least one lubricant in the core portion consists of glyceryl dibehenate.

In any of the oral dosage forms as disclosed above, the oral dosage form can further comprise at least one of an inner seal film sandwiched between the core portion and the controlled membrane film, configured to provide a protective coating for the core portion encapsulated therein; or an outer seal film coating an outer surface of the controlled membrane film.

Herein optionally, at least one of the inner seal film or the outer seal film can comprise at least one film-forming polymer, each selected from the group consisting of hypromellose, hydroxypropyl methylcellulose (HPMC), hydroxypropylcellulose (HPC), carboxymethylcellulose, polyvinylpyrrolidone (PVP), and polyvinyl alcohol and polyethylene glycol (PEG).

According to some embodiments, the inner seal film or the outer seal film can have a ratio of approximately 0.4% to 40% by weight of the oral dosage form.

In the following, more details are provided to the oral dosage form as described above.

In any of the oral dosage forms above, the core portion can further comprise at least one granulation binding polymer. Each of the at least one binding polymer can be selected from hydroxypropyl methylcellulose (HPMC), hydroxyl-propyl cellulose (HPC), hydroxyethyl cellulose (HEC), poly (ethylene) oxide (PEO), polyvinyl alcohol (PVA), povidone (PVP), and co-povidone. Mixtures of the above-mentioned binding agents may also be used. The preferred binding agents are water soluble such as polyvinyl pyrrolidone having an average molecular weight of 25,000 to 3,000,000 g/mol. The binding agent comprises approximately about 0 to about 40% of the total weight of the core and preferably about 3% to about 15% of the total weight of the core. The core should comprise at least one penetration/absorption enhancer. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-big (B-aminoethyl ether-N,N,N,N-tetraacetic acid (EGTA)). The core comprises approximately 0 to about 20% of the absorption enhancer based on the total weight of the core and most preferably about 2% to about 10% of the total weight of the core. In this embodiment the core which comprises the antihyperglycemic drug, the binder which preferably is a pharmaceutically acceptable water soluble polymer and the absorption enhancer is preferably formed by wet granulating the core ingredients and compressing the granules with the addition of a lubricant into a tablet on a rotary press. The core may also be formed by dry granulating the core ingredients and compressing the granules with the addition of a lubricant into tablets or by direct compression. Other commonly known excipients may also be included into the core such as lubricants, pigments or dyes.

In the core portion, the metformin or the pharmaceutically acceptable salt thereof can comprise at least one of metformin hydrochloride, metformin sulfate, metformin phosphate, metformin hydrobromide, metformin salicylate, metformin maleate, metformin benzoate, metformin succinate, metformin ethanesulfonate, metformin fumarate, or metformin glycolate, as disclosed in U.S. Pat. No. 3,174,901. One preferred example of the metformin or the pharmaceutically acceptable salt thereof herein is metformin hydrochloride (metformin HCl), which may have a dosage form of approximately 100-2000 mg. For example, the oral dosage form provided in the oral dosage form may include 100 mg, 250 mg, 500 mg, 750 mg, 1000 mg or 2000 mg, and preferably 250 mg, 500 mg, 750 mg and 1000 mg, of metformin HCl.

When the oral dosage form is tested in a USP Type II apparatus at approximately 50 rpm at approximately 37° C. in 900 ml of a medium having a pH of approximately 6.8, the metformin hydrochloride is configured to exhibit a dissolution profile such that upon contacting the oral dosage form with the medium: less than 30% of the metformin hydrochloride is released at approximately 4 hours; 30-50% of the metformin hydrochloride is released at approximately 8 hours; 40-65% of the metformin hydrochloride is released at approximately 12 hours; and no less than 75% of the metformin hydrochloride is released at approximately 24 hours.

The metformin HCl controlled release oral dosage form is configured to provide a mean maximum plasma concentration (Cₘₐₓ) of metformin from approximately 0.5*X ng/ml to approximately 0.9*X ng/ml, based on administration of the oral dosage form comprising X mg of metformin HCl, wherein X is in a range of approximately 100-1000. In certain embodiment, the controlled release oral dosage form comprises 1000 mg of metformin HCl, the oral dosage form as such is configured to provides a mean maximum plasma concentration (Cₘₐₓ) of the drug that is about 500-900 ng/ml, based on administration of the 1000 mg once-a-day dose of metformin. In another embodiment, the controlled release oral dosage form comprises 500 mg of metformin HCl, the oral dosage form as such is configured to provides a mean maximum plasma concentration (Cₘₐₓ) of the drug that is about 250-450 ng/ml, based on administration of the 500 mg once-a-day dose of metformin.

The metformin HCl controlled release oral dosage form is configured to provide a mean maximum AUC0-t from approximately 7*Y hr*ng/ml to approximately 16*Y hr*ng/ml, based on administration of the oral dosage form comprising Y mg of metformin HCl, wherein Y is in a range of approximately 100-1000. In certain embodiments, the controlled release oral dosage form comprises 1000 mg of metformin HCl, the controlled release dosage form provides a mean AUC0-24 hr that is about 7000-16000 ng.hr/ml, based on administration of a 1000 mg once-a-day dose of metformin. In another embodiment, the controlled release oral dosage form comprises 500 mg of metformin HCl, the controlled release dosage form provides a mean AUC0-24 hr that is about 3500-8000 ng.hr/ml, based on administration of a 500 mg once-a-day dose of metformin.

In any of the embodiments of the oral dosage form as described above, the controlled membrane film may comprise at least one water insouble polymer. Each of the at least one water insouble polymer is selected from a cellulose acetate, or a cellulose acetate phthalate. According to some embodiments, the at least one water insouble polymer in the controlled membrane film of the oral dosage form of the pharmaceutical composition can be cellulose esters, cellulose diesters, cellulose triesters, cellulose ethers, cellulose acetate phthalate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,11210 which are incorporated herein by reference. The most preferred membrane material is cellulose acetate (i.e. “CA”), which can be selected from CA-320S, CA-398-3, CA-398-6, CA-398-10, CA-398-30, CA398-60S, or any combination thereof. Herein, each of CA-320S, CA-398-3, CA-398-6, CA-398-10, CA-398-30, CA398-60S shall be interpreted to have the meaning as well-known by people of ordinary skills in the field, which can be respectively referred to Handbook of Pharmaceutical Execipients. More specifically, CA-320S can be referred to as cellulose acetate molecules with molecular weight around 38,000 g/mol, CA-398-3 can be referred to as cellulose acetate molecules with a molecular weight around 30,000 g/mol, CA-398-6 can be referred to as cellulose acetate molecules with molecular weight around 35,000 g/mol, CA-398-10 can be referred to as cellulose acetate molecules with molecular weight around 40,000 g/mol, CA-398-30 can be referred to as cellulose acetate molecules with molecular weight around 50,000 g/mol, and CA398-60S can be referred to as cellulose acetate molecules with a molecular weight around 60,000 g/mol. In some specific embodiments, the at least one controlling polymer comprises CA-398-10. According to some embodiments of the oral dosage form of the pharmaceutical composition, a relative amount of the at least one controlling polymer in the controlled membrane film of the metformin HCl controlled release oral dosage form can be approximately 1% to 100% of the controlled membrane film by weight. Herein optionally, the relative amount of the at least one controlling polymer can be approximately 1.5% to 7.0% of the tablet by weight. According to some embodiments of the oral dosage form of the pharmaceutical composition, the controlled membrane film further comprises at least one plasticizing agent such as polyethylene glycol, which has a relative amount of approximately 0.1-40% of the controlled membrane film by weight. Herein optionally, the at least one polyglycol can comprise polyethylene glycol (PEG) 6000 or 3350, which is configured to have a relative amount of approximately 0.1-40% of the controlled membrane film by weight.

According to some embodiments of the oral dosage form of the pharmaceutical composition, the at least one passageway is mechanically or optically created in the middle of membrane coated tablets.

As used herein the term passageway includes an aperture, orifice, bore, hole, weakened area or an erodible element such as a gelatin plug that erodes to form an osmotic passageway for the release of the antihyperglycemic drug from the dosage form. A detailed description of the passage-way can be found in U.S. Pat. Nos. such as 3,845,770, 3,916,899, 4,034,758, 4,063,064, 4,077,407, 4.088,864, 4,783,337, and 5.071,607 (the disclosures of which are hereby incorporate by reference). In certain embodiments, the passageway is formed by laser drilling. In preferred embodiments of the invention, the dosage form contains one passageway on each side of each tablet in order provide the desired pharmacokinetic parameters of the formulation.

In any of the embodiments of the oral dosage form as described above, there can be two passageways, which may be arranged on each side of the controlled membrane film. Preferably, each of the two passageways may be arranged within ±5 mm, and preferably within ±2 mm, of a center of each side of the controlled membrane film. Each passageway may have a diameter of approximately 0.30-2.00 mm, and preferably of 0.40-0.60 mm. Each passageway may have a depth of approximately 0.10-2.00 mm, and preferably of 0.30-1.40 mm.

According to certain embodiments, the oral dosage form optionally further comprises an inner seal film, which is sandwiched between the core portion and the controlled membrane film, configured to provide a protective coating for the core portion encapsulated therein.

According to certain embodiments, the oral dosage form optionally further comprises an outer seal film coating an outer surface of the outer portion, which is configured to provide a protective coating for the outer portion encapsulated therein.

According to certain embodiments, the oral dosage form optionally comprises both an inner seal film sandwiched between the core portion and the controlled membrane film and an outer seal film coating an outer surface of membrane coated tablets.

In the above embodiments of the oral dosage form, at least one of the inner seal film or the outer seal film comprises at least one film-forming polymer, each selected from the group consisting of hypromellose, hydroxypropyl methylcellulose (HPMC), hydroxypropylcellulose (HPC), carboxymethylcellulose, polyvinylpyrrolidone (PVP), and polyvinyl alcohol and polyethylene glycol (PEG).

In the above embodiments of the oral dosage form, the inner seal film or the outer seal film has a ratio of approximately 0.4% to 40%, and preferably 2%-10%, by weight of the oral dosage form.

In the above embodiments of the oral dosage form, the inner seal film, the outer seal film, or both, may further comprise at least one of a plasticizers, or a pigment, or a dispersing agent, or an antioxidant.

In any of the embodiments of the oral dosage form of the pharmaceutical composition as described above, the subject can be a human, or a mammalian organism that is afflicted by diabetes.

In a second aspect, the present disclosure further provides a method for manufacturing an oral dosage form of a pharmaceutical composition. The oral dosage form can be the oral dosage form of the composition according to any one of the embodiments as described above in the first aspect.

The method can comprise the following steps (A-C):
(A) preparing a core portion comprising metformin or a pharmaceutically acceptable salt thereof;
(B) coating the core portion with a controlled membrane film, wherein the controlled membrane film is provided with at least one passageway configured to allow the at least one antidiabetic agent to release out of the core portion therethrough;

Optionally, the method comprises a step between step (A) and step (B), comprising:
coating the core portion with an inner seal film, wherein the inner seal film is configured to provide a protective coating for the core portion encapsulated therein.

Optionally, the method comprises a step after step (B), comprising:
coating the membrane coated tablets with an outer seal film, wherein the outer seal film is configured to provide a protective coating for the membrane coating therein.

In step (A) of the method, the core portion comprising at least one antidiabetic agent can be formulated by the following sub-steps:
(1) granulation-by dry granulation, wet granulation or a fluid bed granulation;
(2) milling;
(3) mixing; and
(4) compression.

Optionally, after sub-step (4) of obtaining the core portion, step (A) further comprises a sub-step (5) of coating the core portion with a seal coating solution, wherein the seal coating solution can be made of hypromellose or hydroxypropyl cellulose and polyethylene glycol or other suitable water-soluble material by first dissolving the hypromellose or hydroxypropyl cellulose and polyethylene glycol. The coating solution was then sprayed onto the core tablets using a pan coater. The seal film constitutes about 2%-10% by weight of the oral dosage form. In step (B) of the method, the controlled membrane film may comprise at least one controlling polymer, each optionally selected from a cellulose acetate or cellulose acetate phthalate polymers; and at least one passageway can then be generated in the controlled membrane film.

According to some embodiments of the method, step (B) of coating the core portion with a controlled membrane film comprises the following sub-steps:
(1) preparing a spray suspension, wherein the spray suspension comprises CA-398-10 and PEG 400, 3350, 6000 or 8000; and
(2) coating the core portion with the spray suspension to thereby obtain a coated tablets; and
(3) curing the coated tablets.

In the above embodiments of the method, the above sub-step (3) of curing the coated tablets comprises:
curing the coated tablets for approximately at least one hour at 50° C.

In certain embodiments, each of the at least one passageway is formed by laser drilling. In preferred embodiments of the invention, the oral dosage form contains one passageway on each side of each tablet in order provide the desired pharmacokinetic parameters of the formulation, the orifice should be within ±2 mm of the middle of the tablet, diameter of orifice should be between 0.30 to 0.60 mm, and the depth of orifice should be between 0.10 to 1.40 mm.

In certain embodiments of the method where the membrane coated tablets is further coated with an outer seal film, the outer seal film can be fabricated by the following procedures. Briefly, an outside seal coating solution can be prepared by mixing all the excipients in the required amount of purified water using a suitable homogenizer until the solids are dissolved. The membraned coated tablets can be loaded into a suitable perforated side-vented coating pan with baffles fitted with single or multi spray gun to produce a spray to cover the entire width of the tablet bed; the average weight of warmed uncoated tablet is determined as the initial starting weight; the seal coating solution will be sprayed onto the tablet bed at a suitable spray rate and atomization pressure; spraying will be continued while monitoring the tablet weight until the required weight gain is obtained.

Throughout the disclosure, the term “tablet” is intended to encompass compressed pharmaceutical dosage formulations of all shapes and sizes, whether coated or uncoated.

The term “pharmaceutically acceptable” as used herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The terms “pharmaceutical composition” or “dosage form” as used herein are used interchangeably and are defined to mean a pharmaceutical composition, preparation or system in which doses of medicine or active drug are included. Pharmaceutical compositions or dosage forms can be administered by any route of administration known to the skilled in the art, including but not limited to oral, parenteral, pulmonary, rectal, vaginal, nasal and topical.

The term “dosage form” as it is used herein means at least one unit dosage form of the present invention (e.g. the daily dose of the antihyperglycemic agent can be contained in 2 unit dosage forms of the present invention for single once-a-day administration).

The term “oral dosage form” as used herein is defined to mean a dosage form which is administered by mouth, for absorption through the mucous membranes of the mouth and/or, after swallowing, through the gastrointestinal tract. Such oral dosage forms include but are not limited to solutions, syrups, suspensions, emulsions, gels, powders, granules, capsules, tablets, buccal dosage forms and sublingual dosage forms.

The term “therapeutically effective” when used herein is meant to signify an amount of an antidiabetic agent that can reduce blood glucose levels by approximately the same amount as an immediate release reference standard (e.g., Glucophage) or more, when the controlled release dosage form is orally administered to a human patient on a once-a-day basis.

The term “immediate release” (short as “IR”), is defined for the purpose of this present disclosure as the release of an active drug content from an oral dosage form into the gastrointestinal tract within a short period of time after administration, and typically the plasma drug levels also peak shortly after dosing. Accordingly, the term “immediate release dosage forms” is referred to as dosage forms which exhibit an “immediate release” of the active drug, and thus provide a substantially immediate rate of release of the active drug.

The term “controlled release” (short as “CR”) as referred to in the whole disclosure is considered to be interchangeable with the terms “extended release” (short as “ER”), “prolonged release” (short as “PR”), “sustained release” (short as “SR”), and is defined for the purpose of this present disclosure as the release of the active drug over an extended period of time (e.g. from about 12 hours to about 24 hours) compared to an immediate release dosage form, such that plasma concentrations of the active drug are maintained for a longer time at a therapeutic level, and therapeutic benefit is maintained for a prolonged period. Accordingly, the terms “controlled release dosage forms”, “immediate release dosage forms”, “prolonged release dosage forms”, and/or “sustained release dosage forms”, are referred to as dosage forms which exhibit an “controlled release”, an “immediate release”, a “prolonged release”, and/or a “sustained release” of the active drug.

Throughout the disclosure, the different formulations of Fortamet are referred to as the extended release dosage form of metformin HCI, which is branded in Andrx Labs and other companies, and has been described in U.S. Pat. No. 6,866,866. The Glumetza, 1000 mg is referred to as a dosage form of metformin, branded in Santarus Inc., and has been described in U.S. Pat. No. 7,780,978.

Throughout the disclosure, the relatively term “approximately”, “about”, “around”, or alike, that is behind a number, is referred to as a description of an actual number that is within 5% of the indicated number. In one illustrating example, “approximately 1.00” can be interpreted that the actual number is between 0.95 and 1.05.

### BRIEF DESCRIPTION OF THE DRAWINGS

FIG. 1 is a schematic diagram of an oral dosage form of metformin or a pharmaceutically acceptable salt thereof according to some embodiments of the disclosure.

### DETAILED DESCRIPTION

In the following, with reference to the above-mentioned drawings of various embodiments disclosed herein, the technical solutions of the various embodiments of the disclosure will be described in a clear and fully understandable way. It is noted that the described embodiments represent merely a portion, but not all, of the embodiments of the disclosure. Based on the described embodiments of the disclosure, those ordinarily skilled in the art can obtain other embodiment(s), which shall come within the scope sought for protection by the disclosure.

In the first aspect, the present disclosure provides a dosage form of a pharmaceutical composition that is specifically used for treating a subject with diabetes or prediabetes.

Herein the dosage form is preferably an oral dosage form that is taken by the subject orally, and the subject can be, but is not limited to, a human, and can also be yet another insulin-producing mammal, such as a monkey, a chimpanzee, a dog, a cat, etc.

Regarding the controlled-release formulation of an antidiabetic agent in an oral dosage form of the pharmaceutical composition, it can be realized by arranging the antidiabetic agent at a core of the dosage form, and the core is further coated with a controlled membrane film which is configured to realize a controlled release of the core-residing antidiabetic agent.

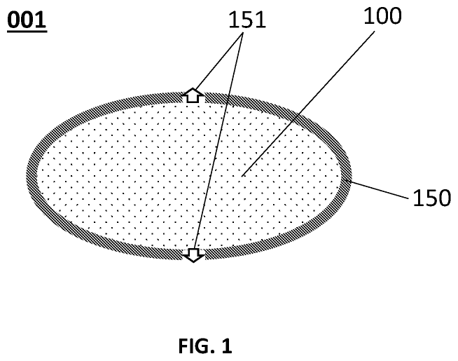

FIG. 1 illustrates a structural diagram of an oral dosage form of the pharmaceutical composition according to certain embodiments of the present disclosure. As illustrated in the figure, the oral dosage form 001 of the pharmaceutical composition substantially comprises a core portion 100 comprising at least one antidiabetic agent.

A controlled membrane film 150 is coated around the core portion 100 to thereby encapsulate the core portion 100. One or more delivery passageways (e.g., orifices, pores, holes, or alike) 151 are arranged on the controlled membrane film 150, which are configured to provide delivery passageways for the at least one antidiabetic agent in the core portion 100 to get out of the controlled membrane film 150, so as to realize a controlled release or an extended release thereof. In FIG. 1, only two delivery passageways 151 are shown, yet it is noted that it is for illustration purposes only, and there is no limitation on the number of delivery passageways arranged in the controlled membrane film 150. Optionally, each of the delivery passageways can be further provided with a soluble plug (not shown in the figure), such as a water soluble material (e.g. gelatin) or an enteric material, which is configured to seal the passageways but can dissolve or leach in a water solution (e.g. GI fluid) to thereby open the delivery passageways to allow the at least one antidiabetic agent in the core portion 100 to release. Herein, the controlled membrane film 150 can comprise from about 1% to about 7%, and preferably from about 2% to about 6%, of the whole oral dosage form in mass weight. In certain preferred embodiments of the disclosure, the dosage form contains two passageways that are configured to provide the desired pharmacokinetic parameters of the formulation.

Herein, the controlled membrane film 150 can be a semipermeable membrane by being permeable to the passage of external fluid such as water and biological fluids and being impermeable to the passage of the antidiabetic drug in the core. As such, the controlled membrane film 150 can comprise at least one water insoluble polymer. Non-limiting examples for the insoluble polymer that can be used for forming the controlled membrane film 150 herein include a cellulose ester, a cellulose diester, a cellulose triester, a cellulose ether, a cellulose ester-ether, cellulose acylate, cellulose diacylate, cellulose triacylate, cellulose acetate, cellulose diacetate, cellulose triacetate, cellulose acetate propionate, or cellulose acetate butyrate, etc. Other suitable polymers are described in U.S. Pat. Nos. 3,845,770, 3,916,899, 4,008,719, 4,036,228 and 4,11210 which are incorporated herein by reference. For example, one membrane material as such can be CA-398-10, i.e., cellulose acetate that comprises an acetyl content of 39.3%-40.3% and is commercially available from Eastman Fine Chemicals.

Herein optionally, the controlled membrane film 150 can comprise at least one insoluble polymer as described above and at least one soluble excipient (i.e., pore-forming agent) which are mixed together with one another. The at least one soluble excipient may optionally include at least one pore-forming agent and/or at least one plasticizer. The major compositions in the controlled membrane film 150 and their ratios are summarized in Table 1.

TABLE 1
Main ingredients in the controlled membrane
film of the oral dosage form.

| Ingredient | Preferred | More Preferred |
|---|---|---|
| Polymer | 50-99% | 75-95% |
| Pore-forming agent | 0-40% | 2-20% |
| Plasticizer | 0-25% | 2-15% |

Note:
the percentages in the table represent the weight percentage of each ingredient, i.e. the percentrage of each ingredient in the controlled membrane film by weight.

Optionally, the controlled membrane film 150 can comprise one or more insoluble polymers as described above and at least one pore-forming agent. The at least one pore-forming agent increases the volume of fluid (water and biological fluid) penetrating into the core to enable the dosage form of the metformin or a pharmaceutically acceptable salt thereof to dispense substantially all of the metformin or a pharmaceutically acceptable salt thereof through the passageway(s) and/or through the porous membrane. The pore-forming agent can be a water-soluble material or an enteric material. Some examples of the preferred materials that are useful as pore-forming agent include sodium chloride, potassium chloride, sucrose, sorbitol, mannitol, polyethylene glycol (PEG), propylene glycol, hydroxypropyl cellulose, hydroxypropyl methylcellulose, hydroxypropyl methylcellulose phthalate, cellulose acetate phthalate, polyvinyl alcohols, methacrylic acid copolymers and mixtures thereof. According to some embodiments, the at least one pore-forming agent may comprise polyethylene glycol (PEG), which can be PEG 400, PEG 3350, PEG 6000, PEG 8000 or any combination thereof. Herein, PEG 400, PEG 3350, PEG 6000, and PEG 800 have the meanings as well-known by people of ordinary skills in the field, which are respectively referred to as polyethylene glycol with an average molecular weight of about 400 g/mol, about 3350 g/mol, about 6000 g/mol, and about 8000 g/mol. The at least one pore-forming agent comprises approximately 0 to about 40% of the total weight of the coating (i.e. the controlled membrane film), most preferably about 2% to about 20% of the total weight of the coating. Upon the oral dosage forms contacting a water solution such as the GI fluid, the pore-forming agent dissolves or leaches from the controlled membrane film 150, thereby forming paths in the controlled membrane film 150 for the fluid to enter the core and dissolve the active ingredient (i.e. metformin or a pharmaceutically acceptable salt thereof).

Optionally yet preferably, the controlled membrane film 150 may also be formed with certain excipients such as at least one plasticizer. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are polyethylene glycol (PEG), propylene glycol, triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating (i.e. the controlled membrane film).

As used herein, the term “passageway” can refer to an opening (e.g. an aperture, orifice, bore, hole, or alike) of the controlled membrane film 150 allowing the release of the at least one antidiabetic agent that is encapsulated in the core portion 100 therethrough, and can also refer to a weakened region or an erodible region (e.g. an erodible plug that erodes in an aqueous environment) in the controlled membrane film 150 which can be induced to form an opening therein to thereby allow release of the metformin or a pharmaceutically acceptable salt thereof therethrough. A detailed description of a “passageway” can be found in U.S. Pat. Nos. such as 3,845,770, 3,916,899, 4,034,758, 4,063,064, 4,077,407, 4,088,864, 4,783,337, and 5,071,607, the disclosures of which are hereby incorporate by reference. In certain embodiments, the passageways of the controlled membrane film 150 in the oral dosage form 001 are formed by mechanical or laser drilling. In other embodiments, the passageways are formed by making an indentation onto the core prior to the membrane coating to form a weakened area of the membrane at the point of the indentation.

Optionally, the oral dosage form of the pharmaceutical composition can further comprise an inner seal film (not shown in FIG. 1), which is sandwiched between the core portion 100 and the controlled membrane film 150. The inner seal film substantially encapsulates the core portion 100, and is configured to provide a sealing means therefor before the core portion 100 is coated with the controlled membrane film 150 so that the characteristics of the active pharmaceutical ingredients included in the core portion of the oral dosage form of the pharmaceutical composition provided in the present disclosure is not influenced during the coating process.

Herein, the inner seal film can comprise at least one film-forming polymer and one or more excipients that are pharmaceutically acceptable. Examples of a film-forming polymer include hydroxypropyl methylcellulose (HPMC), hydroxypropylcellulose (HPC), sodium carboxymethylcellulose, polyvinylpyrrolidone (PVP), polyvinyl alcohol, polyethylene glycol (PEG), hypromellose, or other suitable water-soluble polymer materials. The excipients contained in the outer seal film can similarly include plasticizer(s), pigment(s) (i.e. dye(s) or colorant(s)), dispersing agent(s), and antioxidant(s). The compositions of the plasticizers, pigments, dispersing agents and the antioxidants are known to people killed in the field. Examples of a plasticizer include polyethylene glycol (PEG) grades 400, 3350, 6000, 8000 (i.e. PEG 400, PEG 3350, PEG 6000 and PEG 8000, respectively) and triethyl citrate. An example of a dispersing agent can be hydrated aluminum silicate (Kaolin). Examples of an antioxidant include α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), or butylated hydroxyanisole (BHA), etc. The preferred antioxidant is propyl gallate. Herein the antioxidant serves to prevent oxidative degradation of the core portion of the oral dosage form of the pharmaceutical composition provided in the present disclosure.

Herein, the relative amount of the inner seal film relative to the entire tablet, can vary within the scope of the invention and depend on the desirable drug load, which can range from about 0.5%-40%, and for example, from 2% to 10%, of the tablet dosage form by weight.

In the core portion 100 of the oral dosage form disclosed herein, metformin or a pharmaceutically acceptable salt thereof can consist of a water soluble metformin salt, such as metformin hydrochloride (HCl), which can have a dosage (i.e. weight of metformin HCl) of approximately 100-1500 mg, such as 100 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or 1500 mg. Additionally, the metformin HCl may comprise from about 50% to about 98%, or optionally from about 75% to about 95% (e.g. 80%) of the whole oral dosage form in mass weight. As such, with this substantial osmotic pump formulation provided by the oral dosage form provided herein, metformin or a pharmaceutically acceptable salt thereof can realize a controlled or sustained release after a subject takes the oral dosage form, yet without employing any expanding polymer.

In certain embodiments, the core portion of the controlled release oral dosage form of metformin hydrochloride in the present invention further comprises, besides metformin or a pharmaceutically acceptable salt thereof, at least one binding agent (also known as “binder”), and/or optionally at least one absorption enhancer, and/or optionally at least one lubricant, as listed in Table 2.

TABLE 2
Main ingredients in the core portion of the oral dosage form.

| Ingredient | Optional | Preferred |
|---|---|---|
| Metformin or a | 50-98% | 75-95% |
| pharmaceutically | | |
| acceptable salt thereof | | |
| Binder(s) | 0-40% | 3-15% |
| Absorption Enhancer(s) | 0-20% | 2-10% |
| Lubricant(s) | 0-10% | 1-6% |

Note:
the percentages in the table represent the weight percentage of each ingredient in the core portion, i.e. the percentrage of each ingredient in the core portion by weight.

Herein, a binding agent may be any conventionally known pharmaceutically acceptable binder such as polyvinyl pyrrolidone (also known as “providone” or “polyvidone”, shorted as “PVP”), hydroxypropyl cellulose, hydroxyethyl cellulose, ethylcellulose, polymethacrylate, waxes and the like. Mixtures of the aforementioned binding agents may also be used. Optionally, the binding agent used for the core portion of the metformin oral dosage form can comprise a water soluble molecule such as hydroxypropyl cellulose (such as Klucel-EF, i.e. hydroxypropyl cellulose with a molecular weight of about 80,000 g/mol, or Klucel-HF, i.e. hydroxypropyl cellulose with a molecular weight of about 1,200,000 g/mol), or providone (such as povidone-K30 and K90, i.e. polyvinyl pyrrolidone having an average molecular weight of 50,000 g/mol and 1,000,000 g/mol, respectively). The binding agent(s) can constitute about 0.5 to about 40%, optionally about 3% to about 15%, and preferably from about 4% to about 8% of the total weight of the core portion of the oral dosage form. Further optionally, binding agent(s) can constitute from about 0 to about 30%, optionally from about 2% to about 10%, and preferably from about 5% to about 8%, of the oral dosage form by weight.

The core portion may optionally comprise one or more absorption enhancers. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as calcium acetate, citric acid, cyclodextrins, pentetic acid, etc. The core portion can comprise approximately 0 to about 20% of the absorption enhancer based on the total weight of the core portion, and most preferably about 2% to about 10% of the total weight of the core portion. Optionally, the absorption enhancer(s) used in the core portion of the metformin oral dosage form may compirse sodium lauryl sulfate, which may optionally constitute from about 1% to about 10%, and optionally from about 2% to about 8%, and preferably from about 3% to about 5% of the metformin oral dosage form by weight.

One or more lubricants can be used in the core portion of the oral dosage form provided herein, which can be any type of lubricants commonly known in the art such as magnesium stearate, stearic acid, sodium fumarate, glyceryl behenate, glyceryl dibehenate, etc. Optionally, the lubricant(s) component can comprise from about 0.1% to about 8%, and optionally from about 0.5% to about 6%, of the total weight of the core portion of the oral dosage form. Further optionally, the lubricant(s) component can comprise from about 1% to about 10%, and optionally from about 1% to about 6%, of the oral dosage form by weight.

In addition to the abovementioned binder(s), absorption enhancer(s) and lubricant(s), other excipients that may optionally be included into the core portion of the oral dosage form. Examples can include pigments or dyes.

In certain embodiments of the oral dosage form provided herein, the core tablet (i.e. the core portion) can comprise metformin hydrochloride (i.e. active drug), Povidone (polyvinyl pyrrolidone, or PVP) of USP (United State Pharmacopeia) grade (i.e. binder), sodium lauryl sulfate (i.e. absorption enhancer), and magnesium stearate (i.e. lubricant).

According to certain embodiments of the oral dosage form provided herein, metformin hydrochloride can obtain peak plasma levels from 8 to 20 hours after administration under various conditions. Additionally, the controlled release of metformin can effectively reduce the gastrointestinal (GI) side effects (e.g. diarrhea, nausea, and vomiting, etc.) that are frequently caused by the agent.

The controlled release Metformin HCl oral dosage form provides a mean maximum plasma concentration (Cₘₐₓ) of the drug that is about 500-900 ng/ml, based on administration of a controlled release Metformin HCl oral dosage form providing 1000 mg once-a-day dose of metformin.

The controlled release Metformin HCl dosage form provides a mean AUC₀₋₂₄ₕᵣ that is about 7000-16000 ng.hr/ml, and preferably about 9000-14000 ng·hr/ml, based on administration of a controlled release oral dosage form providing 1000 mg once-a-day dose of metformin.

Because of the above configuration, for the controlled-release dosage form of the Metformin or a pharmaceutically acceptable salt thereof that is suitable for once-a-day administration to human subjects with non-insulin-dependent diabetes mellitus (NIDDM) or prediabetes, the dosage form can control blood glucose levels for up to about 24 hours and can provide controlled release of the drug with a mean time to maximum plasma concentration (Tₘₐₓ) of the drug from 8 to 20 hours after administration and a width at 50% of the height of a mean plasma concentration/time curve of the drug from about 6-15 hours.

According to some embodiments, at least one antidiabetic agent in the core portion 100 may, in addition to Metformin or a pharmaceutically acceptable salt thereof, further comprises one or more other types of antidiabetic agents, which can also realize a controlled release from the oral dosage form after the oral administration, because of the controlled membrane film 150 in the oral dosage form of the pharmaceutical composition.

According to some other embodiments, the at least one antidiabetic agent in the core portion 100 may comprise no biguanide but comprise one or more other types of antidiabetic agents, which can realize a controlled release from the oral dosage form after the oral administration.

In one specific embodiment, the oral dosage form of the pharmaceutical composition comprises an controlled-release form of metformin having a dosage of approximately 100-1200 mg.

According to certain embodiments of the oral dosage form of the pharmaceutical composition provided herein, an outer seal film 200 is arranged to coat an outside surface of the membrane coated tablets as also illustrated in FIG. 1. The outer seal film 200 is configured to provide a protective coating for the oral dosage form, so that the characteristics of the active pharmaceutical ingredients included in the oral dosage form are not influenced by the environment.

Herein, the outer seal film 200 of the oral dosage form can have similar compositions as the inner seal film described above. Briefly, the outer seal film can comprise at least one pharmaceutically acceptable film-forming polymer and one or more pharmaceutically acceptable excipients.

Examples of a film-forming polymer include hydroxypropyl methylcellulose (HPMC), hydroxypropylcellulose (HPC), sodium carboxymethylcellulose, polyvinylpyrrolidone (PVP), polyvinyl alcohol, polyethylene glycol (PEG), hypromellose, or other suitable water-soluble polymer materials. A particular form of HPMC for use as a film-forming polymer is HPMC 2910.

The excipients contained in the outer seal film can similarly include plasticizer(s), pigment(s) (i.e. dye(s) or colorant(s)), dispersing agent(s), and antioxidant(s). Examples of a plasticizer include polyethylene glycol grades 400, 3350, 6000, 8000 and triethyl citrate. An example of a dispersing agent can be hydrated aluminum silicate (Kaolin). Examples of an antioxidant include α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), or butylated hydroxyanisole (BHA), etc. The preferred antioxidant is propyl gallate. Herein the antioxidant serves to prevent oxidative degradation of the core portion of the oral dosage form of the pharmaceutical composition provided in the present disclosure.

Herein, the relative amount of the outer seal film relative to the entire tablet, can vary within the scope of the invention and depend on the desirable drug load, which can range from about 0.5%-40%, and preferably from 2% to 10%, of the tablet dosage form by weight.

Herein, the relative amount of film-forming polymer(s) and plasticizer may vary within the scope of the invention. The plasticizer maybe used independently or as a combination in various ratio. The relative amount of the plasticizer relative to the entire tablet, can vary within the scope of the invention and depend on the desirable drug load. In most cases, the plasticizer can constitute from about 0.1% to 10% by weight of the tablet dosage form, preferably from 1% to 8%. Additionally, the level of antioxidant can constitute from about 0.03% to 0.05%. If hydrated aluminum silicate is used as the dispersing agent, it can constitute from about 0.2% to 5% by weight of the tablet dosage form, preferably from 0.5% to 2%.

The following examples further describe and demonstrate embodiments within the scope of the present invention. These embodiments are given solely for the purpose of illustration and are not intended to be construed as limitations of the present invention as many variations thereof are possible without departing from the spirit and scope of the invention.

According to some preferred embodiments, the oral dosage form of the pharmaceutical composition comprises a controlled-release form of metformin or a pharmaceutically acceptable salt thereof. Herein, more specifically metformin or a pharmaceutically acceptable salt thereof corresponds to the antidiabetic agent arranged at the core portion 100 of the oral dosage form 001 as illustrated in FIG. 1.

One particular solid dosage form as such relates to tablets comprising a ontrolled-release form of metformin hydrochloride. Optionally, the unit dosage strength of the metformin hydrochloride for incorporation into the fixed-dose combination of the present disclosure can be 100, 250, 500, 600, 750 850, 1000 or 1200 milligrams.

The following examples further describe and demonstrate embodiments within the scope of the present disclosure. To be more specific, a total of six embodiments of an oral dosage form of metformin are provided, which substantially take the forms of controlled release metformin hydrochloride (HCl) tablets, termed “metformin HCl controlled release (CR) tablets” or short as “metformin CR tablets” hereinafter). It is to be noted that these embodiments are given solely for the purpose of illustration and are not intended to be construed as limitations of the present disclosure as many variations thereof are possible without departing from the spirit and scope of the invention.

### Embodiment 1 (Metformin Controlled Release Tablets, 1000 mg Oral Dosage Form)

This embodiment illustrates the preparation of an oral dosage form of the pharmaceutical composition, which substantially comprises an osmatic pump controlled release metformin hydrochloride (HCl) tablet that comprises 1000 mg metformin HCl.

To be more specific, the core portion of the controlled release metformin HCl membrane coated tablets were formulated by a fluid bed granulation, where hydroxypropyl cellulose (HPC-EF) was dissolved into water to make approximately 5% solids w/w HPC-EF solution, which was then sprayed on delumped metformin HCl, sorbitol and sodium lauryl sulfate.

The dried granules were dried and milled through a co-mill 0.8 mm. Milled granules were blended with magnesium stearate for approximately 5 minutes at 25 RPM in a V-Blender. The granulation conditions for the core portions of the metformin CR tablets are summarized in Table 3.

TABLE 3
Core portion granulation conditions for the
1000 mg metformin CR tablets (Embodiment 1)

| | |
|---|---|
| Granulation Temperature (° C.) | 45-55 |
| Air Volume (HZ) | 25-45 |
| Atomization Air Pressure (Bar) | 2-3 |
| Fluid Bed Spray Rate (g/min) | 5-10 |

The final blend was compressed on an automatic tablet press into tablets using 10.0×20.5 mm long tooling.

The core tablets were then coated with a controlled membrane film by coating with a solution comprising CA-398-10 (i.e. polymer material for the controlled membrane film) and PEG 6000 (i.e. flux enhancer for the controlled membrane film) in acetone solution, followed by curing for 60 minutes at 50° C. The controlled membrane coating condition is summarized in Table 4.

TABLE 4
Controlled membrane coating conditions for the
1000 mg metformin CR tablets (Embodiment 1)

| | |
|---|---|
| Product Temperature (° C.) | 15-30 |
| Air Volume (HZ) | 35 |
| Atomization Air Pressure (Bar) | 0.9 |
| Spray Rate (g/min) | 10-20 |
| Pan Speed (rpm) | 17-18 |
| Inlet Temperature (° C.) | 20-30 |

The coated tablets were laser drilled two holes (e.g. one hole on each side of the tablet, as illustrated in FIG. 1), and each hole should be within ±5 mm of the middle of the tablet, the diameter of orifice should be between 0.40 to 0.60 mm, and the depth of orifice should be between 0.10 to 2.00 mm.

The coated tablets were then coated with an outer seal film. The seal coating solution used for the outer seal film comprises hypromellose and polyethylene glycol or other suitable water-soluble material by first dissolving the hypromellose and polyethylene glycol, preferably Opadry Clear (YS-1-7006) which is a mixture coating material with hypromellose and polyethylene glycol, in purified water. The Opadry coating solution was then sprayed onto the coated tablets using a pan coater under the following conditions: exhaust air temperature of 38-42° C.; atomization pressure of 28-40 psi; and spray rate of 10-15 ml/min. The Opadry Clear of the coating constitutes about 20-25 mg/tablet.

In Embodiment 1 of the oral dosage form (i.e. Metformin HCl controlled release tablet, 1000 mg) that is obtained by the preparation method as described above, the various compositions for the metformin HCl controlled release tablet, 1000 mg composition are summarized in Table 5.

TABLE 5
Compositions of the 1000 mg metformin
membrane coated tablets (Embodiment 1)

| Component | % w/w |
|---|---|
| Metformin HCl | 82.5 |
| Sodium Lauryl Sulfate | 3.7 |
| Sorbitol | 5.2 |
| HPC-EF | 5.0 |
| Magnesium Stearate | 0.79 |
| Cellulose Acetate | 2.80 |
| Polyethylene glycol | 0.34 |
| Total | 100.0 |

Note:
“% w/w” means the weight percentage of one component over the whole tablet.

Dissolution study was further performed over the 1000 mg metformin CR tablets provided in Embodiment 1. Specifically, multiple tablets prepared above were dissolved in a pH 6.8 buffer, and the dissolution profiles of different tablets at different time points (i.e. 4 hr, 6 hr, 8 hr, 10 hr, 12 hr, 16 hr, 20 hr, and 24h) were measured by USP Apparatus Type II at 50 rpm at 37° C. and summarized in Table 6.

TABLE 6
Dissolution profiles of various tablets of the
1000 mg metformin CR tablets (Embodiment 1)

| | Time Point | | | | | | | |
|---|---|---|---|---|---|---|---|---|
| | 4 hr | 6 hr | 8 hr | 10 hr | 12 hr | 16 hr | 20 hr | 24 hr |
| Tablet #1 | 20.4 | 32.4 | 42.4 | 51.6 | 57.9 | 71.0 | 80.5 | 85.4 |
| (% Diss) | | | | | | | | |
| Tablet #2 | 20.4 | 32.2 | 42.5 | 51.8 | 58.3 | 71.7 | 81.0 | 85.7 |
| (% Diss) | | | | | | | | |
| Tablet #3 | 16.9 | 27.1 | 36.6 | 45.0 | 51.0 | 63.5 | 73.9 | 80.1 |
| (% Diss) | | | | | | | | |
| Tablet #4 | 20.6 | 32.8 | 42.3 | 50.9 | 56.5 | 69.4 | 78.3 | 83.1 |
| (% Diss) | | | | | | | | |
| Tablet #5 | 22.2 | 34.4 | 45.1 | 54.8 | 61.2 | 74.5 | 82.2 | 86.1 |
| (% Diss) | | | | | | | | |
| Tablet #6 | 21.0 | 32.5 | 42.5 | 51.6 | 58.3 | 71.9 | 80.7 | 84.8 |
| (% Diss) | | | | | | | | |
| Mean | 20.3 | 31.9 | 41.9 | 51.0 | 57.2 | 70.3 | 79.4 | 84.2 |
| (% Diss) | | | | | | | | |
| % RSD | 8.7 | 7.8 | 6.7 | 6.3 | 6.0 | 5.3 | 3.8 | 2.7 |

Note:
“% Diss” means percentage of dissolved part of each tablet; “% RSD” means the relative standard deviation (RSD), i.e. the variation, of the dissolution among different tablets, which is expressed as a percentage and defined as the ratio of the dissolution ratio standard deviation to the dissolution ratio mean multiplied by 100.

### Embodiment 2 (Metformin Controlled Release Tablets, 500 mg Oral Dosage Form)

This embodiment illustrates the preparation of an oral dosage form of the pharmaceutical composition, which substantially comprises an osmatic pump controlled release metformin hydrochloride (HCl) tablet that comprises 500 mg metformin HC1.

To be more specific, the core portion of the controlled release metformin HCl membrane coated tablets were formulated by a fluid bed granulation, where povidone (PVP-K90) was dissolved into water to make approximately 7% solids w/w PVP solution, which was then sprayed on delumped metformin HCl and sodium lauryl sulfate.

The dried granules were dried and milled through a co-mill 0.8 mm. Milled granules were blended with magnesium stearate for approximately 5 minutes at 25 RPM in a V-Blender. The granulation conditions for the core portions of the metformin CR tablets are summarized in Table 7.

TABLE 7
Core portion granulation conditions for the
500 mg metformin CR tablets (Embodiment 2)

| | |
|---|---|
| Granulation Temperature (° C.) | 33-45 |
| Air Volume (HZ) | 25-45 |
| Atomization Air Pressure (Bar) | 2-2.5 |
| Fluid Bed Spray Rate (g/min) | 5-10 |

The final blend was compressed on an automatic tablet press into tablets using 12.0×12.0 mm round tooling for the 500 mg formulation.

The core tablets were then coated with a controlled membrane film by coating with a solution comprising CA-398-10 (i.e. polymer material for the controlled membrane film) and PEG 3350 (i.e. pore forming agent/plasticizing agent for the controlled membrane film) in acetone solution, followed by curing for 60 minutes at 50° C. The controlled membrane coating condition is summarized in Table 8.

TABLE 8
Controlled membrane coating conditions for the
500 mg metformin CR tablets (Embodiment 2)

| | |
|---|---|
| Product Temperature (° C.) | 18.4-24.8 |
| Air Volume (HZ) | 35 |
| Atomization Air Pressure (Bar) | 0.9 |
| Spray Rate (g/min) | 14.4-15.9 |
| Pan Speed (rpm) | 17-18 |
| Inlet Temperature (° C.) | 24.3-30 |

The coated tablets were laser drilled two holes (e.g. one hole on each side of the tablet, as illustrated in FIG. 1), and each hole should be within ±5 mm of the middle of the tablet, the diameter of orifice should be between 0.30 to 0.80 mm, and the depth of orifice should be between 0.10 to 2.00 mm.

The coated tablets were then coated with an outer seal film. The seal coating solution used for the outer seal film comprises hypromellose and polyethylene glycol an Opadry material or other suitable water-soluble material by first dissolving the hypromellose and polyethylene glycol, preferably Opadry Clear (YS-1-7006) which is a mixture coating material with hypromellose and polyethylene glycol, in purified water. The Opadry coating solution was then sprayed onto the core tablets using a pan coater under the following conditions: exhaust air temperature of 38-42° C.; atomization pressure of 28-40 psi; and spray rate of 10-15 ml/min. The Opadry Clear of the coating constitutes about 20-25 mg/tablet.

In the oral dosage form (i.e. Metformin HCl controlled release tablet (500 mg) obtained by the preparation method (i.e. EMBODIMENT 2) as described above, the various compositions for the metformin HCl controlled release tablet, 500 mg composition is summarized in Table 9.

TABLE 9
Compositions of the 500 mg metformin membrane
coated tablets (Embodiment 2)

| Materials | % w/w |
|---|---|
| Metformin HCl | 79.8 |
| Povidone (PVP-K90) | 6.4 |
| Sodium Lauryl Sulfate | 4.0 |
| Magnesium Stearate | 4.7 |
| Cellulose Acetate | 4.6 |
| Polyethylene glycol | 0.55 |
| Total | 100.0 |

Dissolution study was further performed over the 500 mg metformin CR tablets provided in Embodiment 2. Specifically, multiple tablets prepared above were dissolved in a pH 6.8 buffer, and the dissolution profiles of different tablets at different time points (i.e. 4 hr, 6 hr, 8 hr, 10 hr, 12 hr, 14 hr, 16 hr, 20 hr, and 24 hr) were measured by USP Apparatus Type II at 50 rpm at 37° C. and summarized in Table 10.

TABLE 10
Dissolution profiles of various tablets of the
500 mg metformin CR tablets (Embodiment 2)

| | Time Point | | | | | | | | |
|---|---|---|---|---|---|---|---|---|---|
| | 4 hr | 6 hr | 8 hr | 10 hr | 12 hr | 14 hr | 16 hr | 20 hr | 24 hr |
| Tablet #1 | 18.5 | 29.1 | 39.7 | 48.9 | 57.9 | 65.6 | 73.0 | 83.1 | 88.3 |
| (% Diss) | | | | | | | | | |
| Tablet #2 | 20.8 | 31.3 | 41.3 | 51.2 | 60.4 | 68.2 | 75.6 | 85.1 | 89.9 |
| (% Diss) | | | | | | | | | |
| Tablet #3 | 17.7 | 27.6 | 37.2 | 46.3 | 54.7 | 62.7 | 70.5 | 81.2 | 87.3 |
| (% Diss) | | | | | | | | | |
| Mean | 19.0 | 29.3 | 39.4 | 48.8 | 57.7 | 65.5 | 73.0 | 83.1 | 88.5 |
| % Diss | | | | | | | | | |
| % RSD | 8.3 | 6.3 | 5.3 | 5.0 | 4.9 | 4.2 | 3.5 | 2.4 | 1.5 |

### Embodiment 3 (Metformin Controlled Release Tablets, 600 mg Oral Dosage Form)

This embodiment illustrates the preparation of an osmatic pump controlled-release (CR) metformin HCl core tablet that comprises 600 mg metformin HCl.

The procedures used were similar to, and can therefore reference to, EMBODIMENT 2 as described above, with certain variations. More specifically, the metformin CR tablet core portion granulation conditions and the controlled membrane coating conditions are summarized in Table 11 and Table 12, respectively.

TABLE 11
Core portion granulation conditions for the
600 mg metformin CR tablets (Embodiment 3)

| | |
|---|---|
| Granulation Temperature (° C.) | 33-45 |
| Air Volume (HZ) | 25-45 |
| Atomization Air Pressure (Bar) | 2-2.5 |
| Fluid Bed Spray Rate (g/min) | 5-10 |

TABLE 12
Controlled membrane coating conditions for the
600 mg metformin CR tablets (Embodiment 3)

| | |
|---|---|
| Product Temperature (° C.) | 18.4-24.8 |
| Air Volume (HZ) | 35 |
| Atomization Air Pressure (Bar) | 0.9 |
| Spray Rate (g/min) | 14.4-15.9 |
| Pan Speed (rpm) | 17-18 |
| Inlet Temperature (° C.) | 24.3-30 |

In the oral dosage form (i.e. Metformin HCl controlled release tablet (600 mg) coated obtained by the preparation method (i.e. EMBODIMENT 3) as described above, the various compositions for the metformin HCl controlled release tablet, 600 mg composition is summarized in Table 13.

TABLE 13
Compositions of the 600 mg metformin membrane
coated tablets (Embodiment 3)

| Materials | % w/w |
|---|---|
| Metformin HCl | 80.0 |
| Povidone K90 | 6.4 |
| Sodium Lauryl Sulfate | 4.0 |
| Magnesium Stearate | 4.8 |
| Cellulose Acetate | 4.3 |
| Polyethylene glycol | 0.51 |
| Total | 100.0 |

Dissolution study was further performed over the 600 mg metformin CR tablets provided in Embodiment 3. Specifically, multiple tablets prepared above were dissolved in a pH 6.8 buffer, and the dissolution profiles of different tablets at different time points (i.e. 4 hr, 6 hr, 8 hr, 10 hr, 12 hr, 14 hr, 16 hr, 20 hr, and 24 hr) were measured by USP Apparatus Type II at 50 rpm at 37° C. and summarized in Table 14.

TABLE 14
Dissolution profiles of various tablets of the
600 mg metformin CR tablets (Embodiment 3)

| | Time Point | | | | | | | | |
|---|---|---|---|---|---|---|---|---|---|
| | 4 hr | 6 hr | 8 hr | 10 hr | 12 hr | 14 hr | 16 hr | 20 hr | 24 hr |
| Tablet #1 | 17.8 | 28.0 | 37.4 | 46.4 | 56.3 | 65.8 | 72.6 | 82.4 | 86.8 |
| (% Diss) | | | | | | | | | |
| Tablet #2 | 17.7 | 28.7 | 38.9 | 48.3 | 57.4 | 65.2 | 72.1 | 81.7 | 87.6 |
| (% Diss) | | | | | | | | | |
| Tablet #3 | 19.1 | 29.8 | 40.0 | 49.2 | 57.9 | 66.0 | 73.3 | 83.1 | 88.3 |
| (% Diss) | | | | | | | | | |
| Tablet #4 | 17.1 | 26.5 | 41.6 | 43.8 | 52.0 | 59.6 | 66.8 | 78.4 | 84.8 |
| (% Diss) | | | | | | | | | |
| Tablet #5 | 17.5 | 27.0 | 36.0 | 44.8 | 53.4 | 60.8 | 67.9 | 79.1 | 84.3 |
| (% Diss) | | | | | | | | | |
| Tablet #6 | 19.5 | 29.9 | 39.6 | 48.8 | 58.0 | 65.6 | 72.7 | 82.8 | 87.7 |
| (% Diss) | | | | | | | | | |
| Mean | 18.1 | 28.3 | 38.9 | 46.9 | 55.8 | 63.8 | 70.9 | 81.2 | 86.6 |
| (% Diss) | | | | | | | | | |
| % RSD | 5.2 | 5.1 | 5.1 | 4.8 | 4.5 | 4.5 | 4.0 | 2.4 | 1.9 |

### Embodiment 4 (Metformin Controlled Release Tablets, 750 mg Oral Dosage Form)

This embodiment illustrates the preparation of an osmatic pump controlled-release (CR) metformin HCl tablet that comprises 750 mg metformin HCl.

The procedures used were similar to, and can therefore reference to, EMBODIMENT 2 as described above, with certain variations. More specifically, the metformin CR tablet core portion granulation conditions and the controlled membrane coating conditions are summarized in Table 15 and Table 16, respectively.

TABLE 15
Core portion granulation conditions for the
750 mg metformin CR tablets (Embodiment 4)

| | |
|---|---|
| Granulation Temperature (° C.) | 33.7-45.4 |
| Air Volume (HZ) | 25-35 |
| Atomization Air Pressure (Bar) | 2-2.5 |
| Fluid Bed Spray Rate (g/min) | 7.54 |

TABLE 16
Controlled membrane coating conditions for the
750 mg metformin CR tablets (Embodiment 4)

| | |
|---|---|
| Product Temperature (° C.) | 17.8-23.4 |
| Air Volume (HZ) | 35 |
| Atomization Air Pressure (Bar) | 0.9 |
| Spray Rate (g/min) | 14.5 |
| Pan Speed (rpm) | 19 |
| Inlet Temperature (° C.) | 22.1-38.0 |

In the oral dosage form (i.e. Metformin HCl controlled release tablet (750 mg) obtained by the preparation method (i.e. EMBODIMENT 4) as described above, the various compositions for the metformin HCl controlled release tablet, 750 mg composition is summarized in Table 17.

TABLE 17
Compositions of the 750 mg metformin membrane
coated tablets (Embodiment 4)

| Materials | % w/w |
|---|---|
| Metformin HCl | 80.3 |
| Povidone K90 | 6.4 |
| Sodium Lauryl Sulfate | 4.0 |
| Magnesium Stearate | 4.8 |
| Cellulose Acetate | 4.0 |
| Polyethylene glycol | 0.48 |
| Total | 100.0 |

Dissolution study was further performed over the 750 mg metformin CR tablets provided in Embodiment 4. Specifically, multiple tablets prepared above were dissolved in a pH 6.8 buffer, and the dissolution profiles of different tablets at different time points (i.e. 4 hr, 6 hr, 8 hr, 10 hr, 12 hr, 14 hr, 16 hr, 20 hr, and 24 hr) were measured by USP Apparatus Type II at 50 rpm at 37° C. and summarized in Table 18.

TABLE 18
Dissolution profiles of various tablets of the
750 mg metformin CR tablets (Embodiment 4)

| | Time Point | | | | | | | | |
|---|---|---|---|---|---|---|---|---|---|
| | 4 hr | 6 hr | 8 hr | 10 hr | 12 hr | 14 hr | 16 hr | 20 hr | 24 hr |
| Tablet #1 | 19.0 | 28.9 | 38.8 | 47.6 | 56.5 | 64.6 | 71.5 | 83.1 | 87.8 |
| (% Diss) | | | | | | | | | |
| Tablet #2% | 19.2 | 28.5 | 38.1 | 47.3 | 54.6 | 62.6 | 68.9 | 78.1 | 85.8 |
| (% Diss) | | | | | | | | | |
| Tablet #3 | 19.3 | 29.1 | 39.0 | 47.7 | 56.4 | 63.9 | 70.6 | 82.3 | 87.7 |
| (% Diss) | | | | | | | | | |
| Mean | 19.2 | 28.8 | 38.6 | 47.5 | 55.8 | 63.7 | 70.3 | 81.1 | 87.1 |
| (% Diss) | | | | | | | | | |
| % RSD | 0.9 | 1.0 | 1.2 | 0.5 | 2.0 | 1.6 | 1.9 | 3.3 | 1.3 |

### Embodiment 5 (Metformin Controlled Release Tablets, 1200 mg Oral Dosage Form)

This embodiment illustrates the preparation of an osmatic pump controlled-release (CR) metformin HCl tablet that comprises 1200 mg metformin HCl.

The procedures used were similar to, and can therefore reference to, EMBODIMENT 1 as described above, with certain variations. More specifically, the metformin CR tablet core portion granulation conditions and the controlled membrane coating conditions are summarized in Table 19 and Table 20, respectively.

TABLE 19
Core portion granulation conditions for the
1200 mg metformin CR tablets (Embodiment 5)

| | |
|---|---|
| Granulation Temperature (° C.) | 33-45 |
| Air Volume (HZ) | 25-45 |
| Atomization Air Pressure (Bar) | 2-2.5 |
| Fluid Bed Spray Rate (g/min) | 5-10 |

TABLE 20
Controlled membrane coating conditions for the
1200 mg metformin CR tablets (Embodiment 5)

| | |
|---|---|
| Product Temperature (° C.) | 18.4-24.8 |
| Air Volume (HZ) | 35 |
| Atomization Air Pressure (Bar) | 0.9 |
| Spray Rate (g/min) | 14.4-15.9 |
| Pan Speed (rpm) | 17-18 |
| Inlet Temperature (° C.) | 24.3-30 |

In the oral dosage form (i.e. Metformin HCl controlled release tablet (1200 mg) obtained by the preparation method (i.e. EMBODIMENT 5) as described above, the various compositions for the metformin HCl controlled release tablet, 1200 mg composition is summarized in Table 21.

TABLE 21
Compositions of the 1200 mg metformin
membrane coated tablets (Embodiment 5)

| Materials | % w/w |
|---|---|
| Metformin HCl | 81.7 |
| Povidone K90 | 6.5 |
| Sodium Lauryl Sulfate | 4.1 |
| Magnesium Stearate | 4.9 |
| Cellulose Acetate | 2.5 |
| Polyethylene glycol | 0.30 |
| Total | 100.0 |

Dissolution study was further performed over the 1200 mg metformin CR tablets provided in Embodiment 5. Specifically, multiple tablets prepared above were dissolved in a pH 6.8 buffer, and the dissolution profiles of different tablets at different time points (i.e. 4 hr, 6 hr, 8 hr, 10 hr, 12 hr, 14 hr, 16 hr, 20 hr, and 24 hr) were measured by USP Apparatus Type II at 50 rpm at 37° C. and summarized in Table 22.

TABLE 22
Dissolution profiles of various tablets of the
1200 mg metformin CR tablets (Embodiment 5)

| | Time Point | | | | | | | | |
|---|---|---|---|---|---|---|---|---|---|
| | 4 hr | 6 hr | 8 hr | 10 hr | 12 hr | 14 hr | 16 hr | 20 hr | 24 hr |
| Tablet #1 | 17.1 | 24.7 | 31.2 | 40.3 | 47.7 | 55.2 | 63.2 | 74.2 | 83.3 |
| (% Diss) | | | | | | | | | |
| Tablet#2 | 17.3 | 25.6 | 33.8 | 42.6 | 49.6 | 57.2 | 63.7 | 74.7 | 79.8 |
| (% Diss) | | | | | | | | | |
| Mean | 17.2 | 25.1 | 32.5 | 41.5 | 48.7 | 56.2 | 63.5 | 74.4 | 81.5 |
| (% Diss) | | | | | | | | | |
| % RSD | 0.9 | 2.5 | 5.7 | 4.0 | 2.8 | 2.5 | 0.6 | 0.5 | 3.1 |

### Embodiment 6 (Metformin Controlled Release Tablets, 1000 mg Oral Dosage Form)

This embodiment illustrates the preparation of an osmatic pump controlled-release (CR) metformin HCl tablet that comprises 1000 mg metformin HCl (1000 composition).

The procedures used were similar to, and can therefore reference to, EMBODIMENT 1 as described above, with certain variations on for example, compositions of the metformin CR tablets. More specifically, the metformin CR tablet core portion granulation conditions and the controlled membrane coating conditions are summarized in Table 23 and Table 24, respectively.

TABLE 23
Core portion granulation conditions for the
1000 mg metformin CR tablets (Embodiment 6)

| | |
|---|---|
| Granulation Temperature (° C.) | 33-45 |
| Air Volume (HZ) | 25-45 |
| Atomization Air Pressure (Bar) | 2-2.5 |
| Fluid Bed Spray Rate (g/min) | 5-10 |

TABLE 24
Controlled membrane coating conditions for the
1000 mg metformin CR tablets (Embodiment 6)

| | |
|---|---|
| Product Temperature (° C.) | 18.4-24.8 |
| Air Volume (HZ) | 35 |
| Atomization Air Pressure (Bar) | 0.9 |
| Spray Rate (g/min) | 14.4-15.9 |
| Pan Speed (rpm) | 17-18 |
| Inlet Temperature (° C.) | 24.3-30 |

In the oral dosage form (i.e. Metformin HCl controlled release tablet (1000 mg) obtained by the preparation method (i.e. EMBODIMENT 6) as described above, the various compositions for the metformin HCl controlled release tablet, 1000 mg composition is summarized in Table 25.

TABLE 25
Compositions of the 1000 mg metformin
membrane coated tablets (Embodiment 6)

| Materials | % w/w |
|---|---|
| Metformin HCl | 82.3 |
| Povidone K90 | 6.6 |
| Sodium Lauryl Sulfate | 4.1 |
| Glyceryl Dibehenate | 4.9 |
| Cellulose Acetate | 1.8 |
| Polyethylene glycol | 0.22 |
| Total | 100.0 |

Dissolution study was further performed over the 1000 mg metformin CR tablets provided in Embodiment 6. Specifically, multiple tablets prepared above were dissolved in a pH 6.8 buffer, and the dissolution profiles of different tablets at different time points (i.e. 4 hr, 6 hr, 8 hr, 10 hr, 12 hr, 14 hr, 16 hr, 20 hr, and 24 hr) were measured by USP Apparatus Type II at 50 rpm at 37° C. and summarized in Table 26.

TABLE 26
Dissolution profiles of various tablets of the
1000 mg metformin CR tablets (Embodiment 6)

| | Time Point | | | | | | | | |
|---|---|---|---|---|---|---|---|---|---|
| | 4 hr | 6 hr | 8 hr | 10 hr | 12 hr | 14 hr | 16 hr | 20 hr | 24 hr |
| Tablet #1 | 26.8 | 37.4 | 47.2 | 55.9 | 63.8 | 70.6 | 77.0 | 84.8 | 90.3 |
| (% Diss) | | | | | | | | | |
| Tablet #2 | 23.6 | 34.2 | 44.5 | 54.1 | 62.7 | 70.1 | 76.2 | 84.2 | 89.2 |
| (% Diss) | | | | | | | | | |
| Tablet #3 | 28.2 | 39.6 | 49.4 | 58.4 | 66.2 | 73.2 | 78.7 | 86.3 | 90.9 |
| (% Diss) | | | | | | | | | |
| Tablet #4 | 25.6 | 36.6 | 46.6 | 55.6 | 64.2 | 71.7 | 77.7 | 86.3 | 91.5 |
| (% Diss) | | | | | | | | | |
| Tablet #5 | 28.9 | 39.5 | 49.2 | 58.0 | 65.6 | 72.8 | 79.4 | 86.3 | 91.0 |
| (% Diss) | | | | | | | | | |
| Tablet #6 | 21.5 | 31.2 | 40.3 | 49.0 | 56.5 | 64.1 | 70.8 | 81.6 | 88.7 |
| (% Diss) | | | | | | | | | |
| Mean | 25.8 | 36.4 | 46.2 | 55.2 | 63.2 | 70.4 | 76.6 | 84.9 | 90.3 |
| (% Diss) | | | | | | | | | |
| % RSD | 10.9 | 8.9 | 7.4 | 6.2 | 5.5 | 4.7 | 4.0 | 2.2 | 1.2 |

In each of the six embodiments above, the in vitro dissolution profile for metformin HCl is determined under the following conditions:

Dissolution Medium: 900 ml, United State Pharmacopeia Phosphate buffer pH 6.8, refer United State Pharmacopeia for the detail procedures of preparation.

Dissolution Method: United State Pharmacopeia Dissolution Test Apparatus Type II at 50 rpm at 37° C. 50 rpm at 37° C.

Detection Method: UV and High Performance Liquid Chromatography methods are developed and used to measure the dissolved Metformin HCl in the collected samples in the tested time points.

Table 27 below compares the in vitro dissolution results for metformin HCl in the six embodiments of the oral dosage form described above and in the three known antidiabetic prescription medicines (Glumetza XR 1000 mg, Fortamet 500 mg, and Fortamet 1000 mg).

TABLE 27
Metformin CR Tablets Dissolution Comparison

| | EMB | EMB | EMB | EMB | EMB | EMB | | | |
|---|---|---|---|---|---|---|---|---|---|
| | ODIM | ODIM | ODIM | ODIM | ODIM | ODIM | | | |
| Time | ENT 1 | ENT 2 | ENT 3 | ENT 4 | ENT 5 | ENT 6 | | | |
| Point | (1000 | (500 | (600 | (750 | (1200 | (1000 | Glumetza | Fortamet | Fortamet |
| (hrs) | mg) | mg | mg) | mg) | mg) | mg) | (1000 mg) | (500 mg) | (1000 mg) |
| 4 | 20.3 | 19.0 | 18.1 | 19.2 | 17.2 | 25.8 | 51 | 36 | 38 |
| 6 | 31.9 | 29.3 | 28.3 | 28.8 | 25.1 | 36.4 | 70 | 51 | 66 |
| 8 | 41.9 | 39.4 | 38.9 | 38.6 | 32.5 | 46.2 | 80 | 73 | 85 |
| 10 | 51.0 | 48.8 | 46.9 | 47.5 | 41.5 | 55.2 | 86 | 86 | 91 |
| 12 | 57.2 | 57.7 | 55.8 | 55.8 | 48.7 | 63.2 | 94 | 94 | 96 |
| 14 | NA | 65.5 | 63.8 | 63.7 | 56.2 | 70.4 | 97 | 99 | 99 |
| 16 | 70.3 | 73.0 | 70.9 | 70.3 | 63.5 | 76.6 | 101 | | |
| 20 | 79.4 | 83.1 | 81.2 | 81.1 | 74.4 | 84.9 | | | |
| 24 | 84.2 | 88.5 | 86.6 | 87.1 | 81.5 | 90.3 | | | |

Note:
Each value for each embodiment and at each time point is the mean value of the dissolution profiles for each embodiment.

As shown in Table 27, compared with the three known antidiabetic prescription medicines, metformin dissolves more slowly for each of the above six embodiments of the oral dosage form provided herein. More specifically, at approximately 4 hr, approximately 17.2-25.8% of metformin in the six embodiments of the oral dosage form disclosed herein is dissolved, whereas approximately 36-51% of metformin in the three prescription medicines is dissolved; at approximately 8 hr, approximately 32.5-46.2% and 73-85% of metformin is dissolved in the six embodiments of the oral dosage form and in the three prescription medicines, respectively; at approximately 12 hr, approximately 48.7-63.2% and 94-96% of metformin is dissolved in the six embodiments of the oral dosage form and in the three prescription medicines, respectively. It is notable that substantially all metformin in the three prescription medicines is completely dissolved at approximately 16 hr, whereas dissolution of metformin in the six embodiments of the oral dosage form disclosed herein is not complete even at approximately 24 hr, when approximately 81.5-90.3% of metaformin is dissolved.

The in vivo plasma bioavailability for metformin of one embodiment (Metformin HCl CR 1000 mg tablets formulation provided in Embodiment 1 of this present disclosure, i.e., “test formulation”) of the oral dosage form of the antidiabetic pharmaceutical compositions in subjects under fed conditions is also examined and compared with the in vivo plasma bioavailability of one known antidiabetic prescription medicine (i.e. Fortamet 1000 mg, shown below as “reference formulation”, “formulation R”, or “R”), which is used as reference control.

Specifically, the plasma concentration of metformin provided in EMBODIMENT 1 (i.e. “formulation T” or “T”) and the reference formulation was determined at different time points (0, 2.0, 3.0, 4.0, 4.5, 5.0, 5.5, 6.0, 6.5, 7.0, 7.5, 8.0, 8.5, 9.0, 9.5, 10.0, 10.5, 11.0, 11.5, 12.0, 14.0, 16.0, 16.0, 20.0, 24.0, 28.0 and 36.0 hours) after the oral dosage form is orally administered to several subjects. The metformin plasma concentration results for the reference formulation (R) and for the test formulation (T) under fed condition are summarized in Table 28 and Table 29, respectively.

It is noted that the plasma concentration of metformin is in the unit of ng/ml; each of the highlighted values in the two tables indicates the maximum plasma concentration (Cₘₐₓ) of metformin for each subject after oral administration of a single dose of the oral dosage form of formulation T or formulation R; and the time point 0 represents the predose concentration of metformin. Herein, “Cₘₐₓ” means the maximum plasma concentration; “Tₘₐₓ” means the time to reach the maximum plasma concentration, “AUC_{0-∞}” means the area under the concentration-time curve from dosing (time 0) to the time of the last measured concentration; “AUC_{0-∞}” means area under the concentration-time curve from time 0 extrapolated to infinite time; “t1/2” means the time required for plasma concentration of a drug to decrease by 50%; “Kₑₗ” means elimination rate constant; and “AUC_%” Extrap_obs (%)” means area under the concentration-time curve from dosing (time t-last measured concentration) to the extrapolated to infinite time to the AUC_{0-∞}%.

TABLE 28
Plasma concentration (ng/ml) profiles of the metformin determined at different
time points after a single dose of the reference formulation (R) is orally
administered to different subjects under fed condition.

| | | | AUC₀₋ₜ | AUC_{0-∞} | | | AUC_% |
|---|---|---|---|---|---|---|---|
| Subject | Cₘₐₓ | Tₘₐₓ | (hr*ng/ | (hr*ng/ | t_{1/2} | Kₑₗ | Extrap_{—} |
| # | (ng/mL) | (hr) | mL) | mL) | (hr) | (1/hr) | obs (%) |
| 1 | 1336.60 | 7.00 | 15285.25 | 16248.59 | 4.67 | 0.148 | 5.93 |
| 3 | 1056.76 | 12.00 | 13500.46 | 15710.73 | 6.04 | 0.115 | 14.07 |
| 4 | 1105.70 | 10.00 | 17278.35 | 17423.51 | 6.45 | 0.108 | 0.83 |
| 5 | 862.67 | 10.00 | 11246.40 | 12442.10 | 4.81 | 0.144 | 9.61 |
| 6 | 1207.49 | 7.50 | 12292.31 | 12660.29 | 3.75 | 0.185 | 2.91 |
| 7 | 1141.08 | 5.00 | 13713.47 | 15833.68 | 7.62 | 0.091 | 13.39 |
| 8 | 1210.36 | 9.00 | 15220.12 | 17479.04 | 6.72 | 0.103 | 12.92 |
| 9 | 1322.94 | 9.02 | 17250.18 | 18308.25 | 4.35 | 0.159 | 5.78 |
| 10 | 930.08 | 7.00 | 12498.78 | 15977.38 | 9.23 | 0.075 | 21.77 |
| 11 | 936.51 | 7.00 | 7283.98 | 7475.56 | 3.72 | 0.186 | 2.56 |
| 12 | 1363.41 | 8.00 | 12274.75 | 12762.97 | 3.92 | 0.177 | 3.83 |
| 13 | 888.74 | 6.00 | 11659.46 | 12440.14 | 4.41 | 0.157 | 6.28 |
| 14 | 1166.97 | 11.00 | 14400.71 | 17385.79 | 6.77 | 0.102 | 17.17 |

Summar Statistics

| | | | | | | | |
|---|---|---|---|---|---|---|---|
| N | 13 | 13 | 13 | 13 | 13 | 13 | 13 |
| Mean | 1117.637 | 8.348 | 13377.248 | 14780.618 | 5.573 | 0.135 | 9.004 |
| SD | 173.0599 | 2.0355 | 2680.3930 | 3054.1733 | 1.7075 | 0.0376 | 6.3893 |
| Min | 862.67 | 5.00 | 7283.98 | 7475.56 | 3.72 | 0.08 | 0.83 |
| Median | 1141.082 | 8.000 | 13500.458 | 15833.681 | 4.814 | 0.144 | 6.275 |
| Max | 1363.41 | 12.00 | 17278.35 | 18308.25 | 9.23 | 0.19 | 21.77 |
| CV % | 15.48 | 24.38 | 20.04 | 20.66 | 30.64 | 27.93 | 70.96 |
| Geometric | 1104.978 | 8.115 | 13095.161 | 14424.608 | 5.349 | 0.130 | 6.595 |
| Mean | | | | | | | |

Note:
“SD” means standard deviation; “CV %” means coefficient of variation, which is equivalent to relative standard deviation (RSD) as defined above.

TABLE 29
Plasma concentration (ng/ml) profiles of the metformin determined at
different time points after a single dose of the test formulation (T)
is orally administered to different subjects under fed condition.

| | | | AUC₀₋ₜ | AUC0-∞ | | | AUC_% |
|---|---|---|---|---|---|---|---|
| Subject | Cₘₐₓ | Tₘₐₓ | (hr*ng/ | (hr*ng/ | t_{1/2} | Kₑₗ | Extrap_{—} |
| # | (ng/mL) | (hr) | mL) | mL) | (hr) | (1/hr) | obs (%) |
| 1 | 1468.84 | 8.50 | 12384.23 | 15186.58 | 6.82 | 0.102 | 18.45 |
| 3 | 831.14 | 14.00 | 10747.34 | 16200.45 | 9.99 | 0.069 | 33.66 |
| 4 | 742.86 | 20.00 | 19048.40 | | | | |
| 5 | 451.52 | 14.00 | 10474.95 | 10655.12 | 6.77 | 0.102 | 1.69 |
| 6 | 866.85 | 14.00 | 10765.06 | 11392.10 | 3.35 | 0.207 | 5.50 |
| 7 | 719.73 | 11.00 | 10568.22 | 12927.58 | 6.24 | 0.111 | 18.25 |
| 8 | 699.90 | 14.00 | 16912.51 | 17179.17 | 6.15 | 0.113 | 1.55 |
| 9 | 985.14 | 11.00 | 16186.73 | 16312.63 | 5.72 | 0.121 | 0.77 |
| 10 | 718.23 | 8.50 | 10143.47 | 14167.26 | 9.13 | 0.076 | 28.40 |
| 11 | 1030.93 | 9.00 | 7298.78 | 7452.36 | 3.01 | 0.231 | 2.06 |
| 12 | 629.10 | 24.00 | 16584.91 | | | | |
| 13 | 678.61 | 14.03 | 9441.89 | 18995.57 | 16.77 | 0.041 | 50.29 |
| 14 | 730.50 | 14.00 | 16897.43 | 16989.77 | 4.86 | 0.143 | 0.54 |

Summar Statistics

| | | | | | | | |
|---|---|---|---|---|---|---|---|
| N | 13 | 13 | 13 | 11 | 11 | 11 | 11 |
| Mean | 811.796 | 13.541 | 12881.069 | 14314.418 | 7.163 | 0.120 | 14.653 |
| SD | 247.5215 | 4.4418 | 3718.9344 | 3413.9619 | 3.8151 | 0.0564 | 16.7978 |
| Min | 451.52 | 8.50 | 7298.78 | 7452.36 | 3.01 | 0.04 | 0.54 |
| Median | 730.496 | 14.000 | 10765.057 | 15186.575 | 6.238 | 0.111 | 5.504 |
| Max | 1468.84 | 24.00 | 19048.40 | 18995.57 | 16.77 | 0.23 | 50.29 |
| CV % | 30.49 | 32.80 | 28.87 | 23.85 | 53.26 | 47.15 | 114.64 |
| Geometric | 781.674 | 12.939 | 12389.271 | 13880.954 | 6.415 | 0.108 | 5.690 |
| Mean | | | | | | | |

As shown in Table 29, the maximum plasma concentration (Tₘₐₓ) of metformin for each subject after oral administration of a single dose of the oral dosage form of formulation T ranges approximately 8-20 hours among different subjects after oral administration, with the mean plasma concentration peaking at 13.5 hours after oral administration.

Furthermore, the key pharmacokinetics (PK) parameters for the Reference (R) formulation and for the Test formulation (T) after oral administration is further summarized in Table 30.

TABLE 30
Pharmacokinetics (PK) parameters for the Reference (R) and Test (T) formulations.

| | Arithmetic Mean ± SD (% CV) | | | |
|---|---|---|---|---|
| Pharmacokinetic | Reference formulation (R) | | Test formulation (T) | |
| Parameters (Units) | (N = 13) | | (N = 13) | |
| Cₘₐₓ (ng/mL) | 1117.637 ± 173.0599 | (15.48%) | 811.796 ± 247.5215 | (30.49%) |
| ^{#}Tₘₐₓ (hr) | 8.000 | (5.00-12.00) | 14.000 | (8.50-24.00) |
| AUC₀₋ₜ (hr*ng/mL) | 13377.248 ± 2680.3930 | (20.04%) | 12881.069 ± 3718.9344 | (28.87%) |
| AUC_{0-∞} (hr*ng/mL) | 14780.618 ± 3054.1733 | (20.66%) | 14314.418 ± 3413.9619 | (23.85%) |
| AUC_% Extrap_obs | 9.004 ± 6.3893 | (70.96%) | 14.653 ± 16.7978 | (114.64%) |

(%)

Note:
^{#}For Tₘₐₓ median (min-max)

It should be noted that these above embodiments and examples of the pharmaceutical composition comprising metformin HCl serve as illustrating examples only and shall not be interpreted as limitations of the scope.

All references cited in the present disclosure are incorporated by reference in their entirety. Although specific embodiments have been described above in detail, the description is merely for purposes of illustration. It should be appreciated, therefore, that many aspects described above are not intended as required or essential elements unless explicitly stated otherwise.

Various modifications of, and equivalent arts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. An oral dosage form of a pharmaceutical composition for managing diabetes or prediabetes in a subject, comprising:
a core portion comprising metformin or a pharmaceutically acceptable salt thereof;
a controlled membrane film encapsulating the core portion, wherein the controlled membrane film is provided with at least one passageway configured to allow the metformin or the pharmaceutically acceptable salt thereof to release out of the core portion therethrough when the oral dosage form is in an aqueous environment;
wherein:
the oral dosage form has a dissolution profile such that upon dissolving in a medium with a pH of approximately 6.8 at approximately 37° C., less than 30% of the metformin or the pharmaceutically acceptable salt thereof is released from the oral dosage form at 4 hours, and less than 92% of the metformin or the pharmaceutically acceptable salt thereof is released from the oral dosage form at 24 hours.

2. The oral dosage form of claim 1, wherein the oral dosage form has a dissolution profile such that upon dissolving in a medium with a pH of approximately 6.8 at approximately 37° C., approximately 30%-50% of the metformin or the pharmaceutically acceptable salt thereof is released from the oral dosage form at 8 hours.

3. The oral dosage form of claim 1 or claim 2, wherein the oral dosage form has a dissolution profile such that upon dissolving in a medium with a pH of approximately 6.8 at approximately 37° C., approximately 45%-70% of the metformin or the pharmaceutically acceptable salt thereof is released from the oral dosage form at 12 hours.

4. The oral dosage form of any one of claims 1-3, wherein the oral dosage form has a dissolution profile such that upon dissolving in a medium with a pH of approximately 6.8 at approximately 37° C., approximately 18-21% of the metformin or the pharmaceutically acceptable salt thereof is released from the oral dosage form at 4 hours, and approximately 82-90% of the metformin or the pharmaceutically acceptable salt thereof is released from the oral dosage form at 24 hours.

5. The oral dosage form of claim 4, wherein the oral dosage form has a dissolution profile such that upon dissolving in a medium with a pH of approximately 6.8 at approximately 37° C., approximately 35%-45% of the metformin or the pharmaceutically acceptable salt thereof is released from the oral dosage form at 8 hours.

6. The oral dosage form of claim 4 or claim 5, wherein the oral dosage form has a dissolution profile such that upon dissolving in a medium with a pH of approximately 6.8 at approximately 37° C., approximately 50%-65% of the metformin or the pharmaceutically acceptable salt thereof is released from the oral dosage form at 12 hours.

7. The oral dosage form of any one of claims 1-6, wherein at a time point between 4 hours and 24 hours after dissolving in a medium with a pH of approximately 6.8 at approximately 37° C., release of the metformin or the pharmaceutically acceptable salt thereof from the oral dosage form has a relative standard deviation (RSD) of no more than 11%.

8. The oral dosage form of claim 7, wherein release of the metformin or the pharmaceutically acceptable salt thereof from the oral dosage form has a relative standard deviation (RSD) of no more than 6%.

9. The oral dosage form of any one of claims 1-6, wherein at 8 hours after dissolving in a medium with a pH of approximately 6.8 at approximately 37° C., release of the metformin or the pharmaceutically acceptable salt thereof from the oral dosage form has a relative standard deviation (RSD) of no more than 7.5%.

10. The oral dosage form of any one of claims 1-6, wherein at 12 hours after dissolving in a medium with a pH of approximately 6.8 at approximately 37° C., release of the metformin or the pharmaceutically acceptable salt thereof from the oral dosage form has a relative standard deviation (RSD) of no more than 6.0%.

11. The oral dosage form of any one of claims 1-10, wherein the oral dosage form realizes a controlled release of the metformin or the pharmaceutically acceptable salt thereof such that upon a single-dose oral administration, the oral dosage form provides a maximum plasma concentration of the metformin or the pharmaceutically acceptable salt thereof in the subject from approximately 8 to 24 hours after administration.

12. The oral dosage form of claim 11, wherein the oral dosage form realizes a controlled release of the metformin or the pharmaceutically acceptable salt thereof such that upon a single-dose oral administration, the oral dosage form provides a maximum plasma concentration of the metformin or the pharmaceutically acceptable salt thereof in the subject at a mean of approximately 13.5 hours and standard deviation of approximately 4.4 hours after administration.

13. The oral dosage form of any one of claims 1-12, wherein the oral dosage form realizes a controlled release of the metformin or the pharmaceutically acceptable salt thereof such that upon a single-dose oral administration, the oral dosage form provides a mean maximum plasma concentration (Cₘₐₓ) of metformin from approximately 0.5*X ng/ml to approximately 0.9*X ng/ml, based on administration of the oral dosage form comprising X mg of metformin HCl, wherein X is in a range of approximately 100-1000.

14. The oral dosage form of any one of claims 1-12, wherein the oral dosage form provides a mean maximum AUC₀₋ₜ from approximately 7*Y hr*ng/ml to approximately 16*Y hr*ng/mL, based on administration of the oral dosage form comprising Y mg of metformin HCl, wherein Y is in a range of approximately 100-1000.

15. The oral dosage form of any one of claims 1-14, wherein the controlled membrane film comprises at least one water insoluble polymer, each selected from a group consisting of a cellulose ester, a cellulose diester, a cellulose triester, a cellulose ether, a cellulose ester-ether, cellulose acylate, cellulose diacylate, cellulose triacylate, cellulose acetate, cellulose diacetate, cellulose triacetate, cellulose acetate propionate, and cellulose acetate butyrate.

16. The oral dosage form of claim 15, wherein the at least one water insoluble polymer in the controlled membrane film comprises cellulose acetate.

17. The oral dosage form of claim 16, wherein the cellulose acetate has an acetyl content of approximately 39.3%-40.3%.

18. The oral dosage form of any one of claims 15-17, wherein the controlled membrane film further comprises at least one pore-forming agent, each selected from a group consisting of sodium chloride, potassium chloride, sucrose, sorbitol, mannitol, polyethylene glycol (PEG), propylene glycol, hydroxypropyl cellulose, hydroxypropyl methylcellulose, hydroxypropyl methylcellulose phthalate, cellulose acetate phthalate, polyvinyl alcohol, and a methacrylic acid copolymer.

19. The oral dosage form of claim 18, wherein the at least one pore-forming agent in the controlled membrane film comprises polyethylene glycol (PEG).

20. The oral dosage form of any one of claims 1-19, wherein the controlled membrane film comprises a weight ratio of 1.8-6.0%.

21. The oral dosage form of any one of claims 1-20, wherein the number of the at least one passageway in the controlled membrane film is two.

22. The oral dosage form of claim 21, wherein the two passageways are arranged on opposing sides of oral dosage form.

23. The oral dosage form of any one of claims 1-22, wherein each of the at least one passageway has a diameter of approximately 0.30-2.00 mm.

24. The oral dosage form of any one of claims 1-23, wherein each of the at least one passageway has a depth of approximately 0.10-2.00 mm.

25. The oral dosage form of any one of claims 1-24, wherein the metformin or a pharmaceutically acceptable salt thereof comprises at least one of metformin hydrochloride, metformin sulfate, metformin phosphate, metformin hydrobromide, metformin salicylate, metformin maleate, metformin benzoate, metformin succinate, metformin ethanesulfonate, metformin fumarate, or metformin glycolate.

26. The oral dosage form of claim 25, wherein the metformin or a pharmaceutically acceptable salt thereof comprises metformin hydrochloride (metformin HCl).

27. The oral dosage form of claim 26, wherein the metformin or a pharmaceutically acceptable salt thereof consists of approximately 100-1200 mg of metformin HCl.

28. The oral dosage form of any one of claims 1-27, wherein the core portion further comprises at least one binder, each selected from a group consisting of polyvinyl pyrrolidone, hydroxypropyl cellulose, hydroxyethyl cellulose, ethylcellulose, polymethacrylate and wax.

29. The oral dosage form of claim 28, wherein the at least one binder in the core portion comprises polyvinyl pyrrolidone, hydroxypropyl cellulose, or a combination thereof.

30. The oral dosage form of claim 29, wherein the at least one binder in the core portion comprises hydroxypropyl cellulose.

31. The oral dosage form of claim 29, wherein the at least one binder in the core portion comprises polyvinyl pyrrolidone having an average molecular weight of 25,000 to 3,000,000 g/mol.

32. The oral dosage form of any one of claims 1-31, wherein the core portion further comprises at least one absorption enhancer, each selected from a group consisting of a fatty acid, a surfactant, a chelating agent and a bile salt.

33. The oral dosage form of claim 32, wherein the at least one absorption enhancer in the core portion comprises a surfactant, selected from a group consisting of sodium lauryl sulfate, sodium taurocholate, and polyoxyethylene 20 sorbitan monooleate.

34. The oral dosage form of claim 33, wherein the at least one absorption enhancer in the core portion comprises sodium lauryl sulfate.

35. The oral dosage form of any one of claims 1-31, wherein the core portion further comprises at least one lubricant, each selected from a group consisting of magnesium stearate, stearic acid, sodium fumarate, glyceryl behenate, and glyceryl dibehenate.

36. The oral dosage form of claim 35, wherein the at least one lubricant in the core portion consists of magnesium stearate, glyceryl dibehenate, or a combination thereof.

37. The oral dosage form of claim 36, wherein the at least one lubricant in the core portion consists of glyceryl dibehenate.

38. The oral dosage form of any one of claims 1-37, further comprising at least one of:
an inner seal film sandwiched between the core portion and the controlled membrane film, configured to provide a protective coating for the core portion encapsulated therein; or
an outer seal film coating an outer surface of the controlled membrane film.

39. The oral dosage form of claim 38, wherein at least one of the inner seal film or the outer seal film comprises at least one film-forming polymer, each selected from the group consisting of hypromellose, hydroxypropyl methylcellulose (HPMC), hydroxypropylcellulose (HPC), carboxymethylcellulose, polyvinylpyrrolidone (PVP), and polyvinyl alcohol and polyethylene glycol (PEG).

40. The oral dosage form of claim 38 or claim 39, wherein the inner seal film or the outer seal film has a ratio of approximately 0.4% to 40% by weight of the oral dosage form.
